(19) United States
(12) Patent Application Publication
(10) Pub. No.: US 2026/0232751 A1
(43) Pub. Date: Aug. 13, 2026

(54) HAIR GROWTH AND ANTI-HAIR GREYING COMPOSITION

(71) Applicants: Debraj SHOME, Mumbai (IN); Rinky KAPOOR, Mumbai (IN)

(72) Inventors: Debraj SHOME, Mumbai (IN); Rinky KAPOOR, Mumbai (IN)

(21) Appl. No.: 19/531,137

(22) Filed: Feb. 5, 2026

Related U.S. Application Data
(63) Continuation-in-part of application No. PCT/IN2023/050876, filed on Sep. 22, 2023.
(63) Continuation-in-part of application No. PCT/IN2025/051587, filed on Sep. 30, 2025.

(30) Foreign Application Priority Data
Nov. 29, 2024 (IN) 202423093628

(51) Int. Cl. A61K 38/06; A61K 9/50; A61K 31/07; A61K 31/197; A61K 31/4188; A61K 31/4415; A61K 31/455; A61K 31/525; A61K 31/7088; A61K 31/714; A61K 33/14; A61K 33/30; A61K 33/34; A61K 33/42; A61K 35/28; A61K 36/258; A61K 38/03; A61K 38/07; A61K 38/18; A61K 38/44; A61K 38/50; A61K 47/02; A61K 47/10; A61K 47/12; A61K 47/18; A61K 47/20; A61K 47/26; A61P 17/14

(52) U.S. Cl. CPC A61K 38/06; A61K 9/5068; A61K 31/07; A61K 31/197; A61K 31/4188; A61K 31/4415; A61K 31/455; A61K 31/525; A61K 31/7088; A61K 31/714; A61K 33/14; A61K 33/30; A61K 33/34; A61K 33/42; A61K 35/28; A61K 36/258; A61K 38/03; A61K 38/07; A61K 38/1833; A61K 38/1858; A61K 38/1866; A61K 38/446; A61K 38/50; A61K 47/02; A61K 47/10; A61K 47/12; A61K 47/183; A61K 47/20; A61K 47/26; A61P 17/14

(57) ABSTRACT

Disclosed herein is an improved pharmaceutical and dermatological hair growth and anti-hair greying composition and to the process of preparation thereof.

### CROSS-REFERENCE TO RELATED APPLICATIONS

This Application is a Continuation-in-Part of:
International Patent Application No. PCT/IN2023/050876, published as WO 2025/032597, filed on Sep. 22, 2023, which claims priority to Indian Patent Application number 202321053030, filed Aug. 8, 2023; and
International Patent Application No. PCT/IN2025/051587, filed on Sep. 30, 2025, which claims priority to Indian Patent Application number 202423093628, filed Nov. 29, 2024.

Each prior international application is incorporated by reference herein.

### TECHNICAL FIELD OF THE INVENTION

The present invention relates to an improved pharmaceutical and dermatological hair growth and anti-hair greying composition and to the process for preparation thereof.

### BACKGROUND AND PRIOR ART

Hair loss is a common condition affecting millions of individuals worldwide, with significant implications for self-esteem and quality of life. It can result from various factors, including genetics, hormonal imbalances, stress, and environmental influences. Androgenetic alopecia (pattern baldness) is the most prevalent cause of hair loss, leading to the progressive thinning of hair and, ultimately, baldness. While many treatment options exist, including topical therapies, oral medications, and surgical interventions, the demand for more effective and minimally invasive solutions continues to rise.

The reasons for hair loss could be heredity, medical conditions, hormonal changes, diet related or a normal part of aging. Certain medications including chemotherapy, HIV/AIDS, hypothyroidism, and malnutrition including iron deficiency cause hair loss.

The common types of hair loss include male- or female-pattern hair loss, Alopecia Areata, and a thinning of hair known as Telogen Effluvium. There are two types of Alopecia (hair loss): non-scarring alopecia and scarring alopecia.

There are various types of hair including terminal hair, vellus hair, and modified terminal hair such as in the eyebrows and eyelashes. Terminal hairs are coarse, pigmented, long hairs in which the bulb of the hair follicle is situated deep in the dermis. Vellus hairs, on the other hand, are fine, thin, non-pigmented short hairs in which the hair bulb is located superficially in the dermis. As alopecia progresses, a transition takes place in the area of approaching baldness wherein the hairs themselves are changing from the terminal to the vellus type. The size of the hair follicles is known to decrease.

Treatments for alopecia include the medications minoxidil (or finasteride) and hair transplant surgery. Alopecia areata may be treated by steroid injections in the affected area, but these need to be frequently repeated to be effective.

Many growth factors have been implicated in controlling different signals in the cycle of hair growth, with some playing major and some playing minor roles. Biomimetic peptides are known to play a role in combating hair loss. These peptides are designed to mimic the natural signaling molecules in the body, mainly growth factors, specifically targeting the hair follicle to promote hair growth. By enhancing cellular communication and stimulating the activity of dermal papilla cells, biomimetic peptides encourage the growth phase (anagen) of the hair cycle, leading to stronger and healthier hair. They also improve the microenvironment around the hair follicle, creating favorable conditions for regeneration.

Exosomes, derived from Human umbilical cord mesenchymal stem cells, animal and plant sources, have emerged as a promising adjunct in regenerative medicine. Exosomes are small extracellular vesicles that carry bioactive molecules, such as growth factors, cytokines, and miRNAs, capable of promoting cellular repair and regeneration. Plant-derived exosomes are especially valued for their biocompatibility and potential to reduce inflammation and oxidative stress, both of which are implicated in hair loss.

The combination of biomimetic peptides with plant-derived exosomes offers a synergistic approach to hair loss treatment. Biomimetic peptides stimulate hair follicle activity, while exosomes enhance cellular communication, repair damaged tissues, and create an optimal environment for hair growth. Together, they provide a potent, non-invasive therapy that addresses both the causes of hair loss and the need for follicular regeneration, offering a comprehensive solution for individuals seeking improved hair density and quality.

Hair greying is a common phenomenon typically associated with aging, characterized by the gradual loss of pigment in hair follicles. This process results from a complex interplay of genetic, environmental, and biochemical factors. Melanin is a natural pigment responsible for determining the coloration of hair, skin, and eyes. Melanin is generated by specialized cells known as melanocytes situated in the basal layer of the epidermis and within the hair follicles. In addition to its involvement in pigmentation, melanin serves a protective purpose by absorbing and dispersing ultraviolet rays from the sun, thereby shielding cells from harm. As the production of melanin decreases with age, the hair undergoes a transition from its original color to grey and ultimately white, resulting in a change in its visual presentation. Additionally, oxidative stress and the accumulation of reactive oxygen species (ROS) contribute to the degradation of melanin, further accelerating the greying process.

Melanogenesis is a multifaceted process influenced by transcription factors including tyrosinase, TRP1 (tyrosinase-related protein-1), and DCT (dopachrome tautomerase; TRP2; tyrosinase-related protein-2). As the expression level of these factors increases, melanin synthesis is stimulated.

Hair loss and hair greying are two prevalent conditions often associated with aging, though they can occur independently or simultaneously. Both conditions share some common underlying mechanisms, such as genetic influences and the impact of oxidative stress, making their simultaneous occurrence a notable feature of the aging process.

There was an unmet need in the art to provide compositions which can prevent hair loss, improve hair growth as well as prevent hair greying with no adverse effects. In light of the same, the present inventors carried out further improvements in the composition and observed that by adding Exosomes and a combination of Peptides along with Growth factors to the hair growth composition and observed that not only was the hair growth improved in terms of Hair density and Shaft diameter of the hair but also the greying of the hair was prevented and grey hair turned to black hair.

### OBJECT OF INVENTION

It is therefore an object of the present invention to provide an improved hair growth composition for effective prevention of hair loss and for stimulation of hair regrowth and further prevent and reverse hair greying which is potently organic, non-toxic, biocompatible and hence favors its prolonged use with minimal to no side effects.

The other objective is to provide the hair growth composition that can serve both as a cosmetic composition as well as dermatological composition with no adverse side effects and to the process for preparation thereof.

### SUMMARY OF THE INVENTION

In an aspect, the present invention provides a pharmaceutical and dermatological hair growth and anti-hair greying composition comprising;
i. Peptides selected from Caffeoyl Tripeptide-1 in an amount of 0.05 mg/L to 10 mg/L; Stem Cell Factor (SCF) including its biomimetic variant sh-Polypeptide-4 in an amount of 0.05 mg/L to 15 mg/L; Caffeoyl sh-Decapeptide-9 in an amount of 0.05 mg/L to 10 mg/L; Tetrapeptide-32 in an amount of 0.05 mg/L to 15 mg/L; Superoxide Dismutase 1 (SOD-1) including its biomimetic variant sh-Polypeptide-60 in an amount of 0.05 mg/L to 15 mg/L; Sirtuin 1 (Sirt-1) including its biomimetic variant sh-Polypeptide-66 in an amount of 0.01 mg/L to 5 mg/L; Alanine/Histidine/Lysine Polypeptide Copper HCl [cu-AHK] in an amount of 0.5 mg/L to 50 mg/L; Biotinoyl sh-Oligopeptide-129 SP [BIO-TDP] in an amount of 10 mg/L to 75 mg/L; alone or mixtures thereof.
ii. Exosomes selected from Panax ginseng Adventitious Root Extracellular Vesicles in an amount of 0.5 mg/L to 50 mg/L; Human Umbilical Mesenchymal Stem Cell Conditioned Media in an amount of 50,000 mg/L to 100,000 mg/L alone or mixtures thereof.
iii. Hepatocyte Growth Factor (HGF) including its biomimetic variant Sh-Polypeptide 62 in an amount of 0.5 mg/L to 50 mg/L;
iv. Platelet-Derived Growth Factor A (PDGFA) including its biomimetic variant Sh-Polypeptide-8 and/or Sh-Polypeptide-59 in an amount of 0.5 mg/L to 500 mg/L;
   along with
v. Vascular endothelial growth factor (Human oligopeptide-11) including its biomimetic analogue Sh-Polypeptide-9 in an amount of 0.01 mg/L-100 mg/L;
vi. Basic fibroblast growth factor (Human oligopeptide-3) including its biomimetic analogue Sh-Polypeptide-1 in an amount of 0.01 mg/L-100 mg/L;
vii. Insulin like growth factor (Human oligopeptide-2) including its biomimetic analogue Sh-Oligopeptide-2 in an amount of 0.01 mg/L-100 mg/L;
viii. Copper tripeptide 1 in an amount of 0.1 mg/L-500 mg/L;
ix. Keratinocyte growth factor including its biomimetic analogue Sh-Polypeptide-3 in an amount of 0.01 mg/L-100 mg/L;
x. Thymosin β4 including its biomimetic analogue Sh-Oligopeptide-4 in an amount of 0.005 mg/L-100 mg/L;
xi. Noggin including its biomimetic variant sh-Polypeptide-13 in an amount of 9-11 mg/L, xii Decapeptide-10 (CG-Keramin2) in an amount of 8.5-10.5 mg/L;
xiii. Octapeptide-2 (Prohairin-β4) in an amount of 0.0025 to 0.005 mg/L;
xiv. Vitamins, minerals, nucleic acids and amino acids in appropriate concentration;
and pharmaceutical and dermatological acceptable excipients.

In another aspect, the pharmaceutical and dermatological hair growth and anti-hair greying composition of the present invention may further comprise other peptides selected from; Elastin scalp peptide (Tetrapetide 32) in the concentration of about 20 ppm; Elastin Hair Peptide (Hexapeptide-12) in the concentration of about 20 ppm; Acetyl Tetrapetide-3 in appropriate concentration; Octapeptide-11 in appropriate concentration; Octapeptide-20 in appropriate concentration; Palmitoyl pentapeptide-3 in appropriate concentration; Sh-Oligopeptide-72 in a concentration of 0.2497 mg/L, Sh-Polypeptide-14 in a concentration of 0.5 mg/L, and the like.

In another aspect, the pharmaceutical and dermatological hair growth and antihair greying composition of the present invention may further comprise other peptides selected from Sh-Oligopeptide-1 in a concentration of 0.2497 mg/L, Sh-Polypeptide-43 in a concentration of 0.2497 mg/L, Sh-Oligopeptide-10 in a concentration of 0.2497 mg/L, Oligopeptide-1 in a concentration of 249.656 mg/L, sr-(Methionyl Sh-Oligopeptide-75 Sh-Polypeptide-60 Hexapeptide-40) in a concentration of 24.96 mg/L, Oligopeptide-1-Sh-Polypepptide-11 in a concentration of 24.96 mg/L, Oligopeptide-1-Sh-Polypeptide-8 in a concentration of 24.96 mg/L, Oligopeptide-1-Sh-Polypeptide-62 in a concentration of 49.93 mg/L contained in the Bone marrow Conditioned Media.

The vitamins are selected from the group consisting of Vitamin A in an amount of 0.01-0.50 mg/L, Vitamin B1 in an amount of 0.01-0.50 mg/L, Vitamin B2 in an amount of 0.01-0.50 mg/L, Vitamin B3 in an amount of 1.0-50 mg/L, Vitamin B5 in an amount of 0.001-0.050 mg/L, Vitamin B6 in an amount of 0.01-0.10 mg/L, Vitamin B7 in an amount of 0.001-0.050 mg/L, Vitamin B12 in an amount of 0.01-0.50 mg/L, Vitamin C in an amount of 10-80 mg/L, Vitamin E in an amount of 0.001-0.050 mg/L, Vitamin K in an amount of 0.001-0.050 mg/L and the like; minerals and mineral salts selected from the group consisting of Calcium salt in an amount of 100-300 mg/L, Sodium salt in an amount of 5000-7000 mg/L, Potassium salt in an amount of 200-600 mg/L, Magnesium salt in an amount of 10-200 mg/L and the like, nucleic acids selected from the group consisting of Adenosine, Cytosine, Guanine and Thymine and essential and non-essential amino acids selected from the group consisting of Taurine, Hydroxyproline, Methionine, Proline, Ornithine, Asparagine, Glutamic acid, Aspartic acid, Cystine, Serine, Glycine, Tyrosine, Phenylalanine, Isoleucine, Tryptophan, Threonine, Leucine, Histidine, Valine, Arginine, Lysine, Alanine and Glutamine.

The nucleic acids each are in the range of 1-50 mg/L, essential and non-essential amino acid in the range of 2-150 mg/L.

The pharmaceutically and dermatologically acceptable excipients include but are not limited to diluents, carriers, buffering agents, humectants, colorants and the like in appropriate amount.

In a further aspect, present invention provides a process for the preparation of the pharmaceutical and dermatological hair growth and anti-hair greying composition comprising;
i. Adding the Peptides selected from Caffeoyl Tripeptide-1 in an amount of 0.05 mg/L to 10 mg/L; Stem Cell Factor (SCF) including its biomimetic variant sh-Polypeptide-4 in an amount of 0.05 mg/L to 15 mg/L; Caffeoyl sh-Decapeptide-9 in an amount of 0.05 mg/L to 10 mg/L; Tetrapeptide-32 in an amount of 0.05 mg/L to 15 mg/L; Superoxide Dismutase 1 (SOD-1) including its biomimetic variant sh-Polypeptide-60 in an amount of 0.05 mg/L to 15 mg/L; Sirtuin 1 (Sirt-1) including its biomimetic variant sh-Polypeptide-66 in an amount of 0.01 mg/L to 5 mg/L; Alanine/Histidine/Lysine Polypeptide Copper HCl [cu-AHK] in an amount of 0.5 mg/L to 50 mg/L; Biotinoyl sh-Oligopeptide-129 SP [BIO-TDP] in an amount of 10 mg/L to 75 mg/L, Exosomes selected from Panax ginseng Adventitious Root Extracellular Vesicles in an amount of 0.5 mg/L to 50 mg/L; Human Umbilical Mesenchymal Stem Cell Conditioned Media in an amount of 50,000 mg/L to 100,000 mg/L; Growth factors selected from Hepatocyte Growth Factor (HGF) including its biomimetic variant Sh-Polypeptide 62 in an amount of 0.5 mg/L to 50 mg/L; Platelet-Derived Growth Factor A (PDGFA) including its biomimetic variant Sh-Polypeptide-8 and/or Sh-Polypeptide-59 in an amount of 0.5 mg/L to 500 mg/L; Vascular endothelial growth factor (Human oligopeptide-11) including its biomimetic analogue Sh-Polypeptide-9 in an amount of 0.01 mg/L-100 mg/L; Basic fibroblast growth factor (Human oligopeptide-3) including its biomimetic analogue Sh-Polypeptide-1 in an amount of 0.01 mg/L-100 mg/L; Insulin like growth factor (Human oligopeptide-2) including its biomimetic analogue Sh-Oligopeptide-2 in an amount of 0.01 mg/L-100 mg/L; Copper tripeptide 1 in an amount of 0.1 mg/L-500 mg/L; Keratinocyte growth factor including its biomimetic analogue Sh-Polypeptide-3 in an amount of 0.01 mg/L-100 mg/L; Thymosin β4 including its biomimetic analogue Sh-Oligopeptide-4 in an amount of 0.005 mg/L-100 mg/L; Noggin including its biomimetic variant sh-Polypeptide-13 in an amount of 9-11 mg/L, Decapeptide-10 (CG-Keramin2) in an amount of 8.5-10.5 mg/L and Octapeptide-2 (Prohairin-β4) in an amount of 0.0025 to 0.005 mg/L; vitamins, minerals, nucleic acids and amino acids in appropriate concentration; along with pharmaceutically and dermatologically acceptable excipients to distilled water at ambient temperature in the disperser set at 750-1000 rpm;
ii. Adjusting the pH and filtering the final product followed by filling the vials.

In another aspect, the process of the present invention may further comprise addition of other peptides selected from Elastin scalp peptide (Tetrapetide 32) in the concentration of about 20 ppm; Elastin Hair Peptide (Hexapeptide-12) in the concentration of about 20 ppm; Acetyl Tetrapetide-3 in appropriate concentration; Octapeptide-11 in appropriate concentration; Octapeptide-20 in appropriate concentration; Palmitoyl pentapeptide-3 in appropriate concentration; Oligopeptide-74 (CG-Grotene) in appropriate concentration; Sh-Oligopeptide-72 in a concentration of 0.2497 mg/L and Sh-Polypeptide-14 in a concentration of 0.5 mg/L and the like and the Botulinum Toxin Type A is in the concentration of about 100 U/ml and the like.

In another aspect, the process of the present invention may further comprise addition of other peptides selected from Sh-Oligopeptide-1 in a concentration of 0.2497 mg/L, Sh-Polypeptide-43 in a concentration of 0.2497 mg/L, Sh-Oligopeptide-10 in a concentration of 0.2497 mg/L, Oligopeptide-1 in a concentration of 249.656 mg/L, sr-(Methionyl Sh-Oligopeptide-75 Sh-Polypeptide-60 Hexapeptide-40) in a concentration of 24.96 mg/L, Oligopeptide-1-Sh-Polypepptide-11 in a concentration of 24.96 mg/L, Oligopeptide-1-Sh-Polypeptide-8 in a concentration of 24.96 mg/L, Oligopeptide-1-Sh-Polypeptide-62 in a concentration of 49.93 mg/L which are contained in the Bone marrow Conditioned Media.

In another aspect, the present hair growth and anti-hair greying composition may be formulated for intradermal, dermaroller or topical administration. The formulation may be available as ready-to-inject sterile glass vial or as a powder-liquid composition in two separate glass vials that have to be reconstituted prior to administration. It may also be available as prefilled syringes and combined with Hyaluronic Acid.

The composition of the present invention exhibits synergistic effect of the growth factors, peptides and exosomes by preventing hair loss, promoting hair growth and preventing/reversing hair greying and thereby improving the bodily appearance of a person.

The composition of the present invention is effective in preventing hair loss by stimulating hair follicles and promoting hair growth, where such hair growth improves the bodily appearance of said person. It is also effective in preventing greying of hair and reversing the greying of hair by increasing melanin biosynthesis in hair follicles.

In yet another aspect, the present invention provides a method for preventing hair loss by stimulating hair follicles and promoting hair growth and improving the bodily appearance of a person comprising administering the pharmaceutical and dermatological hair growth composition in an effective amount to a person in need.

In another aspect, the present invention provides a method for preventing greying of hair and reversing the greying of hair by increasing melanin biosynthesis in hair follicles and elevating the mRNA expression levels of melanin synthesis factors.

In another aspect, the pharmaceutical and dermatological hair growth and anti-hair greying composition may be administered intradermally or applied topically.

In yet another aspect, the pharmaceutical and dermatological hair growth and anti-hair greying composition may be administered through micro-needling techniques such as the nappage technique, through dermaroller, or hair filler inserts.

The hair growth and anti-hair greying composition of the present invention may be co-administered with one or more hair growth promoting compounds selected from the group consisting of minoxidil, minoxidil analogues, minoxidil derivatives, anti-androgens and 5-alpha-reductase inhibitors and such like in appropriate concentration.

In another aspect, the present hair growth and anti-hair greying composition is used for preventing hair loss and promoting hair growth and preventing and reversing hair greying.

In yet another aspect, the present invention provides a medical device in the form of dermaroller, prefilled syringes, vials or hair filler inserts for administering the hair growth composition of the present invention in an effective amount to the subject.

In another aspect, the present invention provides a kit comprising;
a) prefilled syringes or vials or a dermaroller comprising said pharmaceutical or dermatological composition or hair filler insert embedded in hyaluronic acid enriched with said composition; and
b) instruction manual for application.

### BRIEF DESCRIPTION OF THE DRAWINGS

FIG. 1: depicts effect of present hair growth and anti-hair greying composition on melanin synthesis

FIG. 2: depicts effect of present hair growth and anti-hair greying composition on the mRNA levels of melanin synthesis factors.

FIG. 3: depicts effect of the present hair growth and anti-hair greying composition on inhibiting hair loss and stimulating hair growth.

FIG. 4: Mechanism of action of Bioengineered Exosomal Hair Growth Factor Complex (BEHCTM 10).

FIG. 5a: Bioengineered Exosomal Hair Growth Factor Complex Interaction with receptors on Hair follicles in Wnt pathway.

FIG. 5b: Bioengineered Exosomal Hair Growth Factor Complex Interaction with receptors on Hair follicles in Shh pathway.

FIG. 6 shows hair regrowth progression at during and after treatment with biomimetic polypeptide formulations disclosed herein, in conjunction with hair transplant surgery.

FIG. 7 shows the increase in terminal hairs for patients receiving a hair transplant in conjunction with the biomimetic polypeptide formulations.

FIG. 8 shows the increase in hair density for patients receiving a hair transplant in conjunction with the biomimetic polypeptide formulations.

FIG. 9 shows the change in number of vellus hairs for patients receiving a hair transplant in conjunction with the biomimetic polypeptide formulations.

FIG. 10 shows the change in hair shaft diameter for patients receiving a hair transplant in conjunction with the biomimetic polypeptide formulations.

The novel features which are considered characteristic for the invention are set forth in particular in the appended claims. Other aspects of the invention will become evident upon reference to the following detailed description. The invention itself, however, both as to its construction and its method of operation, together with additional objects and advantages thereof, will be best understood from the following description.

### DETAILED DESCRIPTION OF THE INVENTION

In line with the object of present invention, there is provided a hair growth composition for nourishment of hair follicles, for preventing hair loss, stimulating hair regrowth, preventing hair greying and reversing hair greying in mammals.

As used herein the term ‘effective amount’ means an amount which stimulates hair growth associated with hair loss afflictions like male pattern baldness, or insults such as radiation or chemotherapy.

The term ‘growth factor’ relates to a naturally occurring protein capable of stimulating cellular growth, proliferation and cellular differentiation.

The present invention is based on the synergistic effect of the growth factors, peptides and exosomes effective in preventing hair loss, promoting hair growth, preventing greying of hair and reversing the greying of hair by increasing melanin biosynthesis in hair follicles where such hair growth improves the bodily appearance of said person.

In an embodiment, the present invention provides a hair growth and anti-hair greying composition that can stimulate the conversion of vellus hair to terminal hair as well as increase the rate of growth of terminal hair. Further, the hair growth composition of the present invention prevents greying of hair and reverses hair greying.

Accordingly, in an embodiment, the present invention discloses a pharmaceutical and dermatological hair growth and anti-hair greying composition comprising:
i. Peptides selected from Caffeoyl Tripeptide-1 in an amount of 0.05 mg/L to 10 mg/L; Stem Cell Factor (SCF) including its biomimetic variant sh-Polypeptide-4 in an amount of 0.05 mg/L to 15 mg/L; Caffeoyl sh-Decapeptide-9 in an amount of 0.05 mg/L to 10 mg/L; Tetrapeptide-32 in an amount of 0.05 mg/L to 15 mg/L; Superoxide Dismutase 1 (SOD-1) including its biomimetic variant sh-Polypeptide-60 in an amount of 0.05 mg/L to 15 mg/L; Sirtuin 1 (Sirt-1) including its biomimetic variant sh-Polypeptide-66 in an amount of 0.01 mg/L to 5 mg/L; Alanine/Histidine/Lysine Polypeptide Copper HCl [cu-AHK] in an amount of 0.5 mg/L to 50 mg/L; Biotinoyl sh-Oligopeptide-129 SP [BIO-TDP] in an amount of 10 mg/L to 75 mg/L; alone or mixtures thereof.
ii. Exosomes selected from Panax ginseng Adventitious Root Extracellular Vesicles in an amount of 0.5 mg/L to 50 mg/L; Human Umbilical Mesenchymal Stem Cell Conditioned Media in an amount of 50,000 mg/L to 100,000 mg/L alone or mixtures thereof.
iii. Hepatocyte Growth Factor (HGF) including its biomimetic variant Sh-Polypeptide 62 in an amount of 0.5 mg/L to 50 mg/L;
iv. Platelet-Derived Growth Factor A (PDGFA) including its biomimetic variant Sh-Polypeptide-8 and/or Sh-Polypeptide-59 in an amount of 0.5 mg/L to 500 mg/L;
   along with
v. Vascular endothelial growth factor (Human oligopeptide-11) including its biomimetic analogue Sh-Polypeptide-9 in an amount of 0.01 mg/L-100 mg/L;
vi. Basic fibroblast growth factor (Human oligopeptide-3) including its biomimetic analogue Sh-Polypeptide-1 in an amount of 0.01 mg/L-100 mg/L;
vii. Insulin like growth factor (Human oligopeptide-2) including its biomimetic analogue Sh-Oligopeptide-2 in an amount of 0.01 mg/L-100 mg/L;
viii. Copper tripeptide 1 in an amount of 0.1 mg/L-500 mg/L;
ix. Keratinocyte growth factor including its biomimetic analogue Sh-Polypeptide-3 in an amount of 0.01 mg/L-100 mg/L;
x. Thymosin β4 including its biomimetic analogue Sh-Oligopeptide-4 in an amount of 0.005 mg/L-100 mg/L;
xi. Noggin including its biomimetic variant sh-Polypeptide-13 in an amount of 9-11 mg/L, xii. Decapeptide-10 (CG-Keramin2) in an amount of 8.5-10.5 mg/L;
xiii. Octapeptide-2 (Prohairin-β4) in an amount of 0.0025 to 0.005 mg/L;
xiv. Vitamins, minerals, nucleic acids and amino acids in appropriate concentration;
   and pharmaceutical and dermatological acceptable excipients.

In another embodiment, the pharmaceutical and dermatological hair growth and anti-hair greying composition of the present invention may comprise other peptides selected from; Elastin scalp peptide (Tetrapetide 32) in the concentration of 15-25 ppm; Elastin Hair Peptide (Hexapeptide-12) in the concentration of 15-25 ppm; Acetyl Tetrapetide-3 in the concentration of 50-100 ppm; Octapeptide-11 in the concentration of 15-25 ppm; Octapeptide-20 in the concentration of 10-20 ppm; Palmitoyl pentapeptide-3 in the concentration of 50-100; Oligopeptide-74 (CG-Grotene) in the concentration of 15-25 ppm and the like.

The vitamins are selected from the group consisting of Vitamin A in an amount of 0.01-0.50 mg/L, Vitamin B1 in an amount of 0.01-0.50 mg/L, Vitamin B2 in an amount of 0.01-0.50 mg/L, Vitamin B3 in an amount of 1.0-50 mg/L, Vitamin B5 in an amount of 0.001-0.050 mg/L, Vitamin B6 in an amount of 0.01-0.10 mg/L, Vitamin B7 in an amount of 0.001-0.050 mg/L, Vitamin B12 in an amount of 0.01-0.50 mg/L, Vitamin C in an amount of 10-80 mg/L, Vitamin E in an amount of 0.001-0.050 mg/L, Vitamin K in an amount of 0.001-0.050 mg/L and the like; minerals and mineral salts selected from the group consisting of Calcium salt in an amount of 100-300 mg/L, Sodium salt in an amount of 5000-7000 mg/L, Potassium salt in an amount of 200-600 mg/L, Magnesium salt in an amount of 10-200 mg/L and the like, nucleic acids selected from the group consisting of Adenosine, Cytosine, Guanine and Thymine in an amount of 1-50 mg/L and essential and non-essential amino acids selected from the group consisting of Taurine, Hydroxyproline, Methionine, Proline, Ornithine, Asparagine, Glutamic acid, Aspartic acid, Cystine, Serine, Glycine, Tyrosine, Phenylalanine, Isoleucine, Tryptophan, Threonine, Leucine, Histidine, Valine, Arginine, Lysine, Alanine and Glutamine in an amount of 2-150 mg/L.

The pharmaceutical and dermatological acceptable excipients include but are not limited to diluents, carriers, buffering agents, colorants and the like in appropriate amount. Preferably, suitable carriers or diluents include but are not limited to distilled water, physiologic saline, bacteriostatic saline, (saline containing 0.9 mg/ml benzyl alcohol).

In an embodiment, the biomimetic analogues are derived from potato.

The present invention further encompasses the compositions involving one or more growth factors in addition to the composition of invention as mentioned above.

In yet another embodiment, the present invention discloses a pharmaceutical and dermatological hair growth and anti-hair greying composition, comprising:
i. Peptides selected from Caffeoyl Tripeptide-1 in an amount of 1 mg/L; Stem Cell Factor (SCF) including its biomimetic variant sh-Polypeptide-4 in an amount of 1 mg/L; Caffeoyl sh-Decapeptide-9 in an amount of 1 mg/L; Tetrapeptide-32 in an amount of 1 mg/L; Superoxide Dismutase 1 (SOD-1) including its biomimetic variant sh-Polypeptide-60 in an amount of 0.5 mg/L; Sirtuin 1 (Sirt-1) including its biomimetic variant sh-Polypeptide-66 in an amount of 0.10 mg/L; Alanine/Histidine/Lysine Polypeptide Copper HCl [cu-AHK] in an amount of 10 mg/L; Biotinoyl sh-Oligopeptide-129 SP [BIO-TDP] in an amount of 50 mg/L; alone or mixtures thereof.
ii. Exosomes selected from Panax ginseng Adventitious Root Extracellular Vesicles in an amount of 7.5 mg/L; Human Umbilical Mesenchymal Stem Cell Conditioned Media in an amount of 100,000 mg/L alone or mixtures thereof.
iii. Hepatocyte Growth Factor (HGF) including its biomimetic variant Sh-Polypeptide 62 in an amount of 10 mg/L;
iv. Platelet-Derived Growth Factor A (PDGFA) including its biomimetic variant Sh-Polypeptide-8 and/or Sh-Polypeptide-59 in an amount of 50 mg/L;
   along with
v. Vascular endothelial growth factor (Human oligopeptide-11) including its biomimetic analogue Sh-Polypeptide-9 in an amount of 5 mg/L;
vi. Basic fibroblast growth factor (Human oligopeptide-3) including its biomimetic analogue Sh-Polypeptide-1 in an amount of 2 mg/L;
vii. Insulin like growth factor (Human oligopeptide-2) including its biomimetic analogue Sh-Oligopeptide-2 in an amount of 2 mg/L;
viii. Copper tripeptide 1 in an amount of 10 mg/L;
ix. Keratinocyte growth factor including its biomimetic analogue Sh-Polypeptide-3 in an amount of 1 mg/L;
x. Thymosin β4 including its biomimetic analogue Sh-Oligopeptide-4 in an amount of 0.01 mg/L;
xi. Noggin including its biomimetic variant sh-Polypeptide-13 in an amount of 10 mg/L;
xii. Decapeptide-10 (CG-Keramin2) in an amount of 10 mg/L;
xiii. Octapeptide-2 (Prohairin-β4) in an amount of 0.005 mg/L;
xiv. Vitamins, minerals, nucleic acids and amino acids in appropriate concentration;
   and pharmaceutical and dermatological acceptable excipients.

In an embodiment, the above composition may comprise other peptides selected from; Elastin scalp peptide (Tetrapetide 32) in the concentration of about 20 ppm; Elastin Hair Peptide (Hexapeptide-12) in the concentration of about 20 ppm; Botulinum Toxin Type A in the concentration of about 100 U/ml; Acetyl Tetrapetide-3 in appropriate concentration; Octapeptide-11 in appropriate concentration; Octapeptide-20 in appropriate concentration; Palmitoyl pentapeptide-3 in appropriate concentration; Oligopeptide-74 (CG-Grotene) in appropriate concentration; Sh-Oligopeptide-72 in a concentration of 0.2497 mg/L and Sh-Polypeptide-14 in a concentration of 0.5 mg/L and the like.

In another aspect, the pharmaceutical and dermatological hair growth and antihair greying composition of the present invention may further comprise other peptides contained in the Bone marrow Conditioned Media. These other peptides may be selected from Sh-Oligopeptide-1 in a concentration of 0.2497 mg/L, Sh-Polypeptide-43 in a concentration of 0.2497 mg/L, Sh-Oligopeptide-10 in a concentration of 0.2497 mg/L, Oligopeptide-1 in a concentration of 249.656 mg/L, sr-(Methionyl Sh-Oligopeptide-75 Sh-Polypeptide-60 Hexapeptide-40) in a concentration of 24.96 mg/L, Oligopeptide-1-Sh-Polypeptide-11 in a concentration of 24.96 mg/L, Oligopeptide-1-Sh-Polypeptide-8 in a concentration of 24.96 mg/L, and Oligopeptide-1-Sh-Polypeptide-62 in a concentration of 49.93 mg/L

The vitamins in above hair growth and anti-hair greying composition are selected from the group consisting of Vitamin A in an amount of 0.01-0.50 mg/L, Vitamin B1 in an amount of 0.01-0.50 mg/L, Vitamin B2 in an amount of 0.01-0.50 mg/L, Vitamin B3 in an amount of 1.0-50 mg/L, Vitamin B5 in an amount of 0.001-0.050 mg/L, Vitamin B6 in an amount of 0.01-0.10 mg/L, Vitamin B7 in an amount of 0.001-0.050 mg/L, Vitamin B12 in an amount of 0.01-0.50 mg/L, Vitamin C in an amount of 10-80 mg/L, Vitamin E in an amount of 0.001-0.050 mg/L, Vitamin K in an amount of 0.001-0.050 mg/L and the like; minerals and mineral salts selected from the group consisting of Calcium salt in an amount of 100-300 mg/L, Sodium salt in an amount of 5000-7000 mg/L, Potassium salt in an amount of 200-600 mg/L, Magnesium salt in an amount of 10-200 mg/L and the like, nucleic acids selected from the group consisting of Adenosine, Cytosine, Guanine and Thymine in an amount of 1-50 mg/L and essential and non-essential amino acids selected from the group consisting of Taurine, Hydroxyproline, Methionine, Proline, Ornithine, Asparagine, Glutamic acid, Aspartic acid, Cystine, Serine, Glycine, Tyrosine, Phenylalanine, Isoleucine, Tryptophan, Threonine, Leucine, Histidine, Valine, Arginine, Lysine, Alanine and Glutamine in an amount of 2-150 mg/L.

The pharmaceutical and dermatological acceptable excipients include but are not limited to diluents, carriers, buffering agents, colorants, humectants and the like in appropriate amount. Preferably, suitable carriers or diluents include but are not limited to distilled water, physiologic saline, bacteriostatic saline, (saline containing 0.9 mg/ml benzyl alcohol).

Exosomes, small extracellular vesicles, have emerged as promising therapeutic agents for androgenetic alopecia (AGA) due to their ability to modulate key signaling pathways involved in hair follicle growth. They interact with Wnt3a, WIF1 and Sonic Hedgehog (Shh) proteins.

A ligand is a molecule that binds another specific molecule delivering a signal in the process. Ligands interact with proteins in target cells, which are cells that are affected by chemical signals; these proteins are called receptors.

The exosomes and biomimetic peptides of the present invention form a Bioengineered Exosomal Hair Growth Factor Complex (BEHCTM). The Bioengineered Exosomal Hair Growth Factor Complex comprises Bioengineered Exosomes conjugated with Hair Growth Factors (biomimetic peptides) to deliver the synergistic blend for effective follicular regeneration. The bioengineered exosomes, act as nanoparticle-sized delivery vehicles (50-150 nm), to transport the biomimetic growth factors directly to hair follicles. This ensures precise targeting, sustained therapeutic action and effective follicular regeneration, thereby promoting long-lasting results in hair growth.

In another embodiment, the Bioengineered Exosomal Hair Growth Factor Complex uses Targeted Follicular Delivery System, by employing nanoparticlesized exosomes (50-150 nm) to precisely bind to hair follicle receptors such as Wnt3a, WIF1, and Sonic Hedgehog.

These bioengineered exosomes efficiently encapsulate biomimetic polypeptides, ensuring targeted delivery and sustained release of these polypeptides. This mechanism enhances the bioavailability and efficacy of the growth factors, promoting follicular regeneration, reversing follicular damage to a great extent, and stimulating long-lasting hair growth.

The exosomes of the present invention deliver the biomimetic polypeptides to the target cell in 3 different ways:
i. They bind to target cell membranes through ligands expressed on their surfaces, facilitating the ligand-receptor interaction. This ligand-receptor interaction elicits an immune response and mediates hemostasis, angiogenesis.
ii. They transfer their surface proteins and cytoplasms to the target cells through budding and subsequent fusion with the plasma membranes of the target cells.
iii. They aid in the transfer and release of biomimetic polypeptides and in mediating regulatory processes by fusion or internalization of exosomes, thereby helping in the horizontal transfer of proteins and genetic material from one cell to another.

In another embodiment, the present invention provides a bioengineered exosomal hair growth factor complex (BEHCTM) for promoting hair follicle growth and regeneration by activating Wnt3a protein, suppressing WIF1 and activating the Sonic Hedgehog (Shh) signaling pathway comprising:
i. Exosomes in a size range of 50-150 nm; and
ii. Biomimetic polypeptides selected from Sh-Polypeptide-9 (VEGF); ShPolypeptide-1 (bFGF); Sh-Oligopeptide-2 (IGF-1); Sh-Polypeptide-3 (KGF); Sh-Oligopeptide-4 (Thymosin β4); Copper Tripeptide-1; Hepatocyte Growth Factor (HGF); Stem Cell Factor (SCF); and Noggin.

The biomimetic polypeptides of the bioengineered exosomal hair growth factor complex (BEHCTM) have the following functions:
The Sh-Polypeptide-9 (VEGF) stimulates angiogenesis, enhancing blood supply to hair follicles and promoting follicular activity.
The Sh-Polypeptide-1 (bFGF) enhances dermal fibroblast proliferation, supporting the regeneration of hair follicles.
The Sh-Oligopeptide-2 (IGF-1) sustains the anagen phase of hair growth and prevents follicular apoptosis.
The Sh-Polypeptide-3 (KGF) improves follicular resilience against cytotoxic stress, helping maintain follicle health.
The Sh-Oligopeptide-4 (Thymosin β4) stimulates stem cell activation and increases keratinocyte density for improved follicular regeneration.
The Copper Tripeptide-1 inhibits 5-alpha reductase, reducing local DHT levels and inflammation to support follicle survival and hair growth.
The Hepatocyte Growth Factor (HGF) acts as an anti-inflammatory agent and stimulates hair follicle regeneration and growth.
The Stem Cell Factor (SCF) promotes the activation of stem cells and their involvement in hair follicle regeneration, also serving an anti-inflammatory role.
Noggin inhibits certain inhibitory growth factors, promoting follicle survival and regeneration while contributing to anti-inflammatory effects.

In another embodiment, the present invention provides a novel method to promote hair growth by Targeted Follicular Delivery System using the Bioengineered Exosomal Hair Growth Factor Complex of the present invention comprising modulating Wnt3a, WIF1, and Shh signaling pathways.

In another embodiment, the bioengineered exosomes of the present invention promote hair follicle growth and regeneration by activating Wnt3a protein, suppressing WIF1 and activating the Sonic Hedgehog (Shh) signaling pathway.

The bioengineered exosomes help in hair follicle regulation by transporting key signaling molecules such as Wnt3a protein, microRNA miR-22-5p and Sonic Hedgehog (Shh) proteins.

In another embodiment, the exosomes transport Wnt3a protein, which activates the Wnt pathway by binding to Frizzled receptors on hair follicle cells, thereby promoting hair follicle growth and regeneration.

The bioengineered exosomes transport Wnt3a protein or mRNA to hair follicle cells. Upon binding to Frizzled (Fzd) receptors and co-receptors like LRP5/6 on the cell surface, Wnt3a activates the Wnt/β-catenin signaling pathway, thereby promoting hair follicle growth and regeneration.

The bioengineered exosomes also carry microRNAs (miRNAs) that target and downregulate WIF1 which attaches to Fzd receptors, an inhibitor of the Wnt pathway. For instance, miR-22-5p has shown to reduce WIF1 expression, thereby enhancing Wnt signaling and supporting hair follicle regeneration.

In another embodiment, the exosomes transport microRNA miR-22-5p, which downregulates WIF1, an inhibitor of the Wnt pathway, further enhancing Wnt signaling and facilitating hair growth.

The bioengineered exosomes transport Sonic Hedgehog (Shh) proteins to hair follicle cells. Shh binds to Patched1 (PTCH1) receptors on the cell surface, initiating the Shh signaling pathway, which is crucial for hair follicle development and cycling. The binding of Shh to PTCH1 receptor leads to the activation of Smoothened (SMO) and subsequent GLI1 transcription factors, promoting gene expression that supports hair follicle growth and regeneration.

In another embodiment, the exosomes deliver Sonic Hedgehog (Shh) proteins that bind to Patched1 (PTCH1) receptors, contributing to the regulation of hair development.

The Targeted Follicular Delivery System utilizing the Bioengineered Exosomal Hair Growth Factor Complex of the present invention has the following advantages:
1. Exosome-Based Therapy: Utilizing exosomes to deliver Wnt3a or Shh proteins, or to suppress WIF1 through miRNA delivery, can activate these pathways, potentially reversing hair follicle miniaturization and promoting hair regrowth in AGA patients.
2. Targeted Delivery: The Exosomes are engineered to specifically target hair follicle cells, enhancing the efficacy and reducing potential side effects of treatment.

The composition of the present invention may comprise the known growth factors selected from the group consisting of follistatin, Wnt proteins, β catenin proteins, stem cell factor, and platelet derived growth factors in appropriate concentration. In another embodiment, the present hair growth and anti-hair greying composition may be formulated for intradermal, dermaroller or topical administration. The formulation may be available as vials, prefilled syringes.

In an embodiment, the present invention disclose a medical device in the form of dermaroller, prefilled syringes, vials or hair filler inserts for administering the composition of the present invention in an effective amount to the subject.

In another embodiment, the present composition may be available as ready-to-inject sterile glass vial or as a powder-liquid composition in two separate glass vials that have to be reconstituted prior to administration. It may also be available as prefilled syringes and combined with Hyaluronic Acid.

The present hair growth and anti-hair greying composition as described herein above is an enhanced drug delivery system by incorporating the key ingredients of the composition in a targeted Lecithin-Derived Liposomal encapsulation through metered high concentrations (100 nm~) of liposomes. It is formulated using sonication and Micro-fluidizer technology. Lipoencapsulation/Liposomal derivatives are Lecithin-Derived Liposomes. High concentrations (100 nm~) of liposomes are formulated using sonication and Microfluidizer technology.

In yet another embodiment, the hair growth and anti-hair greying composition of the present invention is prepared by a process comprising;
i. Adding the Peptides selected from Caffeoyl Tripeptide-1 in an amount of 0.05 mg/L to 10 mg/L; Stem Cell Factor (SCF) including its biomimetic variant sh-Polypeptide-4 in an amount of 0.05 mg/L to 15 mg/L; Caffeoyl sh-Decapeptide-9 in an amount of 0.05 mg/L to 10 mg/L; Tetrapeptide-32 in an amount of 0.05 mg/L to 15 mg/L; Superoxide Dismutase 1 (SOD-1) including its biomimetic variant sh-Polypeptide-60 in an amount of 0.05 mg/L to 15 mg/L; Sirtuin 1 (Sirt-1) including its biomimetic variant sh-Polypeptide-66 in an amount of 0.01 mg/L to 5 mg/L; Alanine/Histidine/Lysine Polypeptide Copper HCl [cu-AHK] in an amount of 0.5 mg/L to 50 mg/L; Biotinoyl sh-Oligopeptide-129 SP [BIO-TDP] in an amount of 10 mg/L to 75 mg/L, Exosomes selected from Panax ginseng Adventitious Root Extracellular Vesicles in an amount of 0.5 mg/L to 50 mg/L; Human Umbilical Mesenchymal Stem Cell Conditioned Media in an amount of 50,000 mg/L to 100,000 mg/L; Growth factors selected from Hepatocyte Growth Factor (HGF) including its biomimetic variant Sh-Polypeptide 62 in an amount of 0.5 mg/L to 50 mg/L; Platelet-Derived Growth Factor A (PDGFA) including its biomimetic variant Sh-Polypeptide-8 and/or Sh-Polypeptide-59 in an amount of 0.5 mg/L to 500 mg/L; Vascular endothelial growth factor (Human oligopeptide-11) including its biomimetic analogue Sh-Polypeptide-9 in an amount of 0.01 mg/L-100 mg/L; Basic fibroblast growth factor (Human oligopeptide-3) including its biomimetic analogue Sh-Polypeptide-1 in an amount of 0.01 mg/L-100 mg/L; Insulin like growth factor (Human oligopeptide-2) including its biomimetic analogue Sh-Oligopeptide-2 in an amount of 0.01 mg/L-100 mg/L; Copper tripeptide 1 in an amount of 0.1 mg/L-500 mg/L; Keratinocyte growth factor including its biomimetic analogue Sh-Polypeptide-3 in an amount of 0.01 mg/L-100 mg/L; Thymosin β4 including its biomimetic analogue Sh-Oligopeptide-4 in an amount of 0.005 mg/L-100 mg/L; Noggin including its biomimetic variant sh-Polypeptide-13 in an amount of 9-11 mg/L, Decapeptide-10 (CG-Keramin2) in an amount of 8.5-10.5 mg/L and Octapeptide-2 (Prohairin-β4) in an amount of 0.0025 to 0.005 mg/L; vitamins, minerals, nucleic acids and amino acids in appropriate concentration; along with pharmaceutically and dermatologically acceptable excipients to distilled water at ambient temperature in the disperser set at 750-1000 rpm;
ii. Adjusting the pH and filtering the final product followed by filling the vials.

In another embodiment, above process may further comprise addition of other peptides selected from Elastin scalp peptide (Tetrapetide 32) in the concentration of about 20 ppm; Elastin Hair Peptide (Hexapeptide-12) in the concentration of about 20 ppm; Acetyl Tetrapetide-3 in appropriate concentration; Octapeptide-11 in appropriate concentration; Octapeptide-20 in appropriate concentration; Palmitoyl pentapeptide-3 in appropriate concentration; Oligopeptide-74 (CG-Grotene) in appropriate concentration; Sh-Oligopeptide-72 in a concentration of 0.2497 mg/L, Sh-Polypeptide-14 in a concentration of 0.5 mg/L, and the like.

In another embodiment, the process of the present invention may further comprise addition of other peptides contained in the Bone marrow Conditioned Media. These other peptides may be selected from Sh-Oligopeptide-1 in a concentration of 0.2497 mg/L, Sh-Polypeptide-43 in a concentration of 0.2497 mg/L, Sh-Oligopeptide-10 in a concentration of 0.2497 mg/L, Oligopeptide-1 in a concentration of 249.656 mg/L, sr-(Methionyl Sh-Oligopeptide-75 Sh-Polypeptide-60 Hexapeptide-40) in a concentration of 24.96 mg/L, Oligopeptide-1-Sh-Polypeptide-11 in a concentration of 24.96 mg/L, Oligopeptide-1-Sh-Polypeptide-8 in a concentration of 24.96 mg/L, Oligopeptide-1-Sh-Polypeptide-62 in a concentration of 49.93 mg/L.

The vitamins in above process are selected from the group consisting of Vitamin A in an amount of 0.01-0.50 mg/L, Vitamin B1 in an amount of 0.01-0.50 mg/L, Vitamin B2 in an amount of 0.01-0.50 mg/L, Vitamin B3 in an amount of 1.0-50 mg/L, Vitamin B5 in an amount of 0.001-0.050 mg/L, Vitamin B6 in an amount of 0.01-0.10 mg/L, Vitamin B7 in an amount of 0.001-0.050 mg/L, Vitamin B12 in an amount of 0.01-0.50 mg/L, Vitamin C in an amount of 10-80 mg/L, Vitamin E in an amount of 0.001-0.050 mg/L, Vitamin K in an amount of 0.001-0.050 mg/L and the like; minerals and mineral salts selected from the group consisting of Calcium salt in an amount of 100-300 mg/L, Sodium salt in an amount of 5000-7000 mg/L, Potassium salt in an amount of 200-600 mg/L, Magnesium salt in an amount of 10-200 mg/L and the like, nucleic acids selected from the group consisting of Adenosine, Cytosine, Guanine and Thymine in an amount of 1-50 mg/L and essential and non-essential amino acids selected from the group consisting of Taurine, Hydroxyproline, Methionine, Proline, Ornithine, Asparagine, Glutamic acid, Aspartic acid, Cystine, Serine, Glycine, Tyrosine, Phenylalanine, Isoleucine, Tryptophan, Threonine, Leucine, Histidine, Valine, Arginine, Lysine, Alanine and Glutamine in an amount of 2-150 mg/L.

In another embodiment, the pharmaceutical and dermatological acceptable excipients include but are not limited to diluents, carriers, buffering agents, colorants, humectants and the like in appropriate amount. Preferably, suitable carriers or diluents include but are not limited to distilled water, physiologic saline, bacteriostatic saline, (saline containing 0.9 mg/ml benzyl alcohol).

The composition of the present invention exhibits synergistic effect of the growth factors, peptides and exosomes by preventing hair loss, promoting hair growth and preventing/reversing hair greying.

The composition of the present invention is effective in preventing hair loss by stimulating hair follicles and promoting hair growth, where such hair growth improves the bodily appearance of said person. It is also effective in preventing greying of hair and reversing the greying of hair by increasing melanin biosynthesis in hair follicles.

The pharmaceutical and dermatological hair growth composition of the present invention is used to prevent hair loss by stimulating hair follicles and promoting hair growth and prevent or reverse hair greying by increasing melanin biosynthesis in hair follicles and improve the bodily appearance of a person.

The composition of the present invention is stable and can be stored at room temperature (below 25° C.). The composition of the present invention may also be packaged as prefilled syringes and as a hair filler.

The prefilled syringe containing the composition is a 1 ml syringe with a needle of 31 gauge. The barrel of the syringe is marked with graduations, each syringe consisting of 40 such graduations and each graduation corresponding to one unit. One unit is equivalent to 0.025 mL of the formulation.

The present invention disclose a hair filler insert embedded in hyaluronic acid enriched with present composition for enhanced delivery at the site of action.

The hair growth and anti-hair greying composition of the present invention may be administered to a subject in a manner which will result in an effective delivery of the amount or dose of the ingredients to the area where hair growth or reversal of hair greying is desired.

In another embodiment, the present invention discloses a method of preventing hair loss by stimulating hair follicles and promoting hair growth and improving the bodily appearance of a person comprising administering the pharmaceutical and dermatological hair growth and anti-hair greying composition in an effective amount to a person in need.

In another embodiment, the present invention provides a method for preventing greying of hair and reversing the greying of hair and improving the bodily appearance of a person comprising administering the pharmaceutical and dermatological hair growth and anti-hair greying composition of the present invention in an effective amount to a person in need.

Accordingly, the method of administration of said pharmaceutical and dermatological hair growth and anti-hair greying composition to a subject comprises:
a. cleaning the scalp of the subject with alcohol swab or surgical spirit;
b. treating the affected area of the scalp with pharmaceutical and dermatological hair growth and anti-hair greying composition of the present invention in an effective stimulatory amount to treat or prevent alopecia and prevent or reverse hair greying.

The hair growth and anti-hair greying composition of the present invention may be administered intradermally.

In another embodiment, the present invention provides a method for promoting hair follicle growth and regeneration by activating Wnt3a protein, suppressing WIF1 and activating the Sonic Hedgehog (Shh) signaling pathway comprising:
i. Administering the Bioengineered Exosomal Hair Growth Factor Complex system via injection, or through microneedling; and
ii. Enabling targeted delivery to the hair bulb and activation of regenerative pathways.

The composition is administered into the hair loss affected scalp through standard micro-needling techniques such as the nappage technique, through dermaroller application, hair filler inserts.

In another embodiment, the present invention discloses a device for administering the pharmaceutical and dermatological composition comprising a dermaroller, prefilled syringes, vials or hair filler inserts.

In yet another embodiment, the present invention discloses a kit comprising;
a) prefilled syringes or vials or a dermaroller comprising the pharmaceutical or dermatological composition or hair filler insert embedded in hyaluronic acid enriched with the present composition; and
b) instruction manual for application.

In another embodiment, the present composition can be co-administered with one or more hair growth promoting compounds selected from the group consisting of minoxidil, minoxidil analogs, minoxidil derivatives, anti-androgens and 5 alpha-reductase inhibitors. The one or more hair growth promoting compounds mentioned above may be co-administered in the form of oral, liquid or topical administration.

In another embodiment, the hair growth promoting compounds which are selected from the group consisting of minoxidil, minoxidil analogues, minoxidil derivatives, anti-androgens and 5-alpha-reductase inhibitors may be co-prescribed with the pharmaceutical composition.

In an embodiment, the pharmaceutical and dermatological hair growth and anti-hair greying composition of the present invention may be formulated as intradermal, dermaroller and topical application.

In another embodiment, the present invention relates to a method of preventing hair greying thereby improving the bodily appearance of a person comprising administering to the scalp of subject a hair growth composition comprising Exosomes, Peptides, plurality of growth factors including their biomimetic analogues, Copper tripeptide 1, Noggin, Decapeptide-10 (CG-Keramin2), Octapeptide-2 (Prohairin-β4), along with a nourishing complex of vitamins, minerals, amino acids, nucleic acids together with one or more pharmaceutically and dermatologically acceptable excipients in an amount effective to treat or to prevent hair loss by stimulating hair follicles and promoting hair growth and preventing hair greying by increasing melanin biosynthesis in hair follicles.

The alopecia that may be treated includes androgenetic alopecia, alopecia areata, female pattern baldness and secondary alopecia.

The method of treating alopecia and preventing hair greying and improving the bodily appearance of a person includes the step of introducing or applying an effective amount of hair growth composition into a subject once in 2 to 6 weeks, preferably about once in 3 weeks.

The pharmaceutical and dermatological hair growth and anti-hair greying composition of the present invention increases superficial cutaneous circulation in the subject thereby inhibiting the action of 5-α-reductase enzyme and thereby preventing hair loss by stimulating hair follicles and promoting hair growth.

In an embodiment, the composition of the present invention may be co-administered with one or more hair growth promoting compounds selected from the group consisting of minoxidil, minoxidil analogs, minoxidil derivatives and anti-androgens.

The hair growth and anti-hair greying composition of this invention may be used to stimulate hair growth in humans afflicted with androgenetic alopecia. Thus, these molecules may be administered in order to stimulate hair growth, thereby eliminating or reducing the severity of hair loss and/or the speed at which alopecia progresses. Other hair loss afflictions which can be treated include androgenetic alopecia, alopecia areata, female pattern baldness and secondary alopecia, and hair loss secondary to chemotherapy, radiation, post-COVID19 hair loss etc. (secondary alopecia). In the case of secondary alopecia, these may be used in advance of the hair loss insults such as chemotherapy or radiation regimens, to stimulate hair growth prior to the insults, thereby resulting in reduced amount of hair loss resulting therefrom. The pharmaceutical and dermatological composition increases superficial cutaneous circulation in the subject, inhibits action of 5-α-reductase enzyme and thereby prevents hair loss by stimulating hair follicles and promoting hair growth.

The hair growth and anti-hair greying composition of this invention may be used to prevent greying of hair and reverse grey hair in individuals by increasing melanin biosynthesis in hair follicles and elevating the mRNA expression levels of melanin synthesis factors.

The invention can be better understood by the following non-limiting examples. The examples given are mere an illustration of the instant invention and should not be construed as limiting the scope of the present invention in any manner.

### EXAMPLES

### Example 1: Hair Growth and Anti Hair Greying Composition

TABLE 1
Hair growth and Anti-Hair greying composition

| | | Concentration | | |
|---|---|---|---|---|
| No. | Ingredients Name | (mg/L) | | Function |
| 1 | Caffeoyl Tripeptide-1 | 1 | | Skin Protecting |
| 2 | HGF or its biomimetic | 10 | | Antioxidant, |
| | variant sh-Polypeptide | | | Hair Conditioning, |
| | 62 | | | Skin Conditioning |
| 3 | PDGFA (Platelet- | 50 | | Humectant, |
| | Derived Growth Factor | | | Skin Conditioning |
| | A) or its biomimetic | | | |
| | variant sh-Polypeptide-8 | | | |
| | and/or Sh-Polypeptide-59 | | | |
| 4 | SCF (Stem Cell | 1 | | Antioxidant, |
| | Factor) or its | | | Hair Conditioning, |
| | biomimetic variant | | | Skin Conditioning |
| | sh-Polypeptide-4 | | | |
| 5 | SOD-1 (Superoxide | 0.5 | | Skin Protecting |
| | Dismutase 1) or its | | | |
| | biomimetic variant | | | |
| | sh-Polypeptide-60 | | | |
| 6 | Sirt-1 (Sirtuin 1) or its | 0.1 | | Antioxidant, |
| | biomimetic variant | | | Hair Conditioning, |
| | sh-Polypeptide-66 | | | Skin Conditioning |
| 7 | Alanine/Histidine/ | 10 | | Skin Conditioning |
| | Lysine Polypeptide | | | |
| | Copper HCl | | | |
| | [cu-AHK] | | | |
| 8 | Caffeoyl | 1 | | Skin Conditioning |
| | sh-Decapeptide-9 | | | |
| | [Phyto-NEP] | | | |
| 9 | Tetrapeptide-32 | 1 | | Hair Conditioning, |
| | [ESP/Elastin | | | Skin Protecting |
| | Scalp Peptide] | | | |
| 10 | Biotinoyl | 50 | | Skin Conditioning |
| | sh-Oligopeptide-129 | | | |
| | SP [BIO-TDP] | | | |
| 11 | Panax Ginseng | 7.5 | | Skin Conditioning |
| | Adventitious Root | | | |
| | Extracellular Vesicles | | | |
| 12 | Human Umbilical | 100,000 | | Skin Conditioning |
| | Mesenchymal Stem | | | |
| | Cell Conditioned | | | |
| | Media | | | |
| 13 | VEGFA | 5 | | Skin Conditioning |
| | (sh-Polypeptide-9) | | | |
| 14 | bFGF | 2 | | Skin Conditioning |
| | (sh-Polypeptide-1) | | | |
| 15 | IGF-1 | 2 | | SKIN CONDITIONING, |
| | (sh-Oligopeptide-2) | | | SKIN PROTECTING |
| 16 | KGF-1 | 1 | | SKIN CONDITIONING |
| | (sh-Polypeptide-3) | | | |
| 17 | TMB4 | 0.01 | | SKIN CONDITIONING |
| | (sh-Oligopeptide-4) | | | |
| 18 | Copper Tripeptide-1 | 10 | | SKIN CONDITIONING |
| 19 | Noggin or its | 10 | | Skin Protecting |
| | biomimetic variant | | | |
| | sh-Polypeptide-13 | | | |
| 20 | Decapeptide-10 | 10 | | |
| | (CG-Keramin2) | | | |
| 21 | Octapeptide-2 | 0.005 | | |
| | (Prohairin-β4) | | | |
| 22 | 1,2-Hexanediol | q.s | | Solvent |
| 23 | Water | q.s | | Antiplaque, |
| | | | | Skin Conditioning, |
| | | | | Solvent |
| 24 | Calcium Chloride | 5.83 | mg/L | Astringent, Viscosity |
| | | | | Controlling |
| 25 | Copper Sulfate | 0.000065 | mg/L | Skin Conditioning |
| 26 | Ferric Nitrate | 0.0025 | mg/L | Not Reported |
| 27 | Ferric Sulfate | 0.02 | mg/L | Humectant |
| 28 | Potassium Chloride | 15.59 | mg/L | Viscosity Controlling |
| 29 | Magnesium Chloride | 1.43 | mg/L | Viscosity Controlling |
| 30 | Magnesium Sulfate | 2.44 | mg/L | Bulking, |
| | | | | Hair Conditioning, |
| | | | | Viscosity Controlling |
| 31 | Sodium Chloride | 349.78 | mg/L | Bulking, Masking, |
| | | | | Oral Care, |
| | | | | Viscosity Controlling |
| 32 | Sodium Bicarbonate | 60.00 | mg/L | Abrasive, Buffering, |
| | | | | Deodorant, Oral Care, |
| | | | | Skin Protecting |
| 33 | Sodium Phosphate | 3.13 | mg/L | Buffering |
| 34 | Disodium Phosphate | 3.55 | mg/L | Anticorrosive, Buffering, |
| | | | | Masking |
| 35 | Zinc Sulfate | 0.02 | mg/L | Antimicrobial, Antiplaque, |
| | | | | Astringent, Oral Care |
| 36 | Glucose | 157.55 | mg/L | Humectant |
| 37 | Linoleic Acid | 0.0021 | mg/L | Antistatic, Cleansing, |
| | | | | Emollient, Hair |
| | | | | Conditioning, Skin |
| | | | | Conditioning, Surfactant |
| 38 | Thioctic Acid | 0.01 | mg/L | Antioxidant |
| 39 | Sodium Pyruvate | 2.75 | mg/L | Skin Conditioning |
| 40 | Alanine | 0.22 | mg/L | Antistatic, |
| | | | | Hair Conditioning, |
| | | | | Masking, |
| | | | | Skin Conditioning |
| 41 | Arginine HCl | 7.38 | mg/L | Skin Conditioning |
| 42 | Asparagine | 0.37 | mg/L | Antistatic, |
| | | | | Hair Conditioning, |
| | | | | Masking, |
| | | | | Skin Conditioning |
| 43 | Aspartic Acid | 0.33 | mg/L | Antistatic, |
| | | | | Hair Conditioning, |
| | | | | Masking, |
| | | | | Skin Conditioning |
| 44 | Cysteine | 0.88 | mg/L | Antioxidant, |
| | | | | Antistatic, |
| | | | | Hair Conditioning, |
| | | | | Hair Waving |
| | | | | Or Straightening, |
| | | | | Masking, |
| | | | | Reducing |
| 45 | Cystine | 1.56 | mg/L | Antistatic, |
| | | | | Hair Conditioning, |
| | | | | Masking |
| 46 | Glutamic Acid | 0.37 | mg/L | Antistatic, |
| | | | | Hair Conditioning, |
| | | | | Humectant |
| 47 | Glutamine | 13.25 | mg/L | Antistatic, |
| | | | | Hair Conditioning, |
| | | | | Skin Conditioning |
| 48 | Glycine | 0.94 | mg/L | Antistatic, Buffering, |
| | | | | Hair Conditioning, |
| | | | | Skin Conditioning |
| 49 | Histidine HCl | 1.57 | mg/L | Hair Conditioning, |
| | | | | Skin Conditioning |
| 50 | Isoleucine | 2.72 | mg/L | Antistatic, |
| | | | | Hair Conditioning, |
| | | | | Skin Conditioning |
| 51 | Leucine | 2.95 | mg/L | Antistatic, |
| | | | | Hair Conditioning, |
| | | | | Skin Conditioning |
| 52 | Lysine HCl | 4.56 | mg/L | Hair Conditioning, |
| | | | | Skin Conditioning |
| 53 | Methionine | 0.86 | mg/L | Antistatic, |
| | | | | Hair Conditioning, |
| | | | | Skin Conditioning |
| 54 | Phenylalanine | 1.77 | mg/L | Hair Conditioning, |
| | | | | Masking, |
| | | | | Skin Conditioning |
| 55 | Proline | 0.86 | mg/L | Hair Conditioning, |
| | | | | Skin Conditioning |
| 56 | Serine | 1.31 | mg/L | Antistatic, |
| | | | | Hair Conditioning, |
| | | | | Masking, |
| | | | | Skin Conditioning |
| 57 | Threonine | 2.67 | mg/L | Antistatic, |
| | | | | Hair Conditioning, |
| | | | | Hair Waving |
| | | | | Or Straightening |
| 58 | Tryptophan | 0.45 | mg/L | Antistatic, |
| | | | | Hair Conditioning, |
| | | | | Masking |
| 59 | Tyrosine | 2.79 | mg/L | Antistatic, |
| | | | | Hair Conditioning, |
| | | | | Masking, |
| | | | | Skin Conditioning |
| 60 | Valine | 2.64 | mg/L | Antistatic, |
| | | | | Hair Conditioning, |
| | | | | Masking, |
| | | | | Skin Conditioning |
| 61 | Biotin | 0.000175 | mg/L | Antiseborrhoeic, |
| | | | | Hair Conditioning, |
| | | | | Skin Conditioning |
| 62 | Calcium | 0.11 | mg/L | Antistatic, |
| | Pantothenate | | | Hair Conditioning |
| 63 | Folic Acid | 0.13 | mg/L | Skin Conditioning |
| 64 | Inositol | 0.63 | mg/L | Antistatic, |
| | | | | Hair Conditioning, |
| | | | | Humectant |
| 65 | Niacinamide | 0.10 | mg/L | Smoothing |
| 66 | Pyridoxine HCl | 0.10 | mg/L | Antistatic, |
| | | | | Hair Conditioning, |
| | | | | Skin Conditioning |
| 67 | Riboflavin | 0.01 | mg/L | Cosmetic Colorant, |
| | | | | Skin Conditioning |
| 68 | Thiamine HCl | 0.11 | mg/L | Masking, |
| | | | | Skin Conditioning |
| 69 | Thymidine | 0.02 | mg/L | Skin Conditioning |
| 70 | Cyanocobalamin | 0.03 | mg/L | Skin Conditioning |
| 71 | Adenosine | 400.00 | mg/L | Skin Conditioning |
| 72 | Ferric Nitrate | 0.0025 | mg/L | Humectant |

TABLE 2
Product Specification:

| Test Item | | Specifications | Test Result | | Judgement |
|---|---|---|---|---|---|
| Sense Index | APPEARANCE | transparent | transparent | | suitability |
| | | solution | solution | | |
| | COLOUR | colorless | colorless | | suitability |
| | ODOUR | Characteristic | Characteristic | | suitability |
| | | odor | odor | | |
| | Volume | 5 ml | 5.2 | ml | suitability |
| | | (>97%) | | | |
| Microbiological | Aerobic Bacterial | Max. 100 cfu/g | 0 | cfu/g | suitability |
| Index | Count | | | | |
| | Yeast Mould | Max. 100 cfu/g | 0 | cfu/g | suitability |
| | Count | | | | |

| | | | |
|---|---|---|---|
| Pseudomonas | Absence | Absence | suitability |
| aeruginosa | | | |
| Staphylococcus | Absence | Absence | suitability |
| aureus | | | |
| Escherichia coli | Absence | Absence | suitability |

| | |
|---|---|
| Note on storage | Avoid direct sunlight and store at room temperature. |

### Example 2: Preparation of the Hair Growth and Anti-Hair Greying Composition

1) The solvent i.e. water at a temperature of 25° C. was added with the active ingredients and excipients of the composition (provided in Table 1) to the disperser;
2) The disperser was set at 800 rpm for 30 minutes;
3) The pH was adjusted and a 500 MESH micron filter system was used to filter the final product;
4) This was followed by filling, closing and labeling of the vials.

### Example 3: Assessment to Determine Improvement in Hair Loss

Videomicroscopic photographs were carried out with a proscope digital hand-held camera, at a fixed position in the central area of the scalp from 15 and 20 cm posterior to the glabella. At each set position, images were taken to calculate hair counts per cm2. All videomicroscopic images were analyzed for difference in vellus hair count, terminal hair count, and hair shaft diameter, using specialized software.

TABLE 3
Videomicroscopic assessment showing vellus and terminal hair count
(15 cm and 20 cm) and hair shaft diameter post-treatment

| | Mean at | Mean at | Mean at | |
|---|---|---|---|---|
| | baseline | 4^{th} session | 8^{th} session | δ |
| Vellus hair count | 51.14 | 40.65 | 40.98 | −12.16*** |
| at 15 cm | | | | |
| Vellus hair count | 43.68 | 46.32 | 41.85 | −6.83** |
| at 20 cm | | | | |
| Terminal hair count | 162.23 | 174.25 | 181.78 | +15.95*** |
| at 15 cm | | | | |
| Terminal hair count | 182.78 | 189.93 | 191.75 | +8.07*** |
| at 20 cm | | | | |
| Hair shaft diameter | 28.65 | 27.21 | 33.12 | +4.47* |
| at 15 cm (μm) | | | | |
| Hair shaft diameter | 27.54 | 31.89 | 34.15 | +4.61* |
| at 20 cm (μm) | | | | |

Note:
Paired t test. *p < 0.05, **p < 0.01, ***p < 0.001; δ1 - Mean at eight sessions - mean at baseline; δ2 - Mean at four sessions - mean at baseline.

### Example 4

A standardized photographic technique was used to take photo-graphs of frontal, vertex, and temporo-parietal areas of the scalp at baseline, fourth session, and eighth session. These were assessed by two blinded clinicians and graded on the scale 0-10, with 0 showing no growth and 10 indicating thick hair growth. The mean score was calculated and analyzed.

TABLE 4

| | | 4th | 8th | Post 2 months |
|---|---|---|---|---|
| Reviewer | Baseline | Session | Session | after 8th session |
| Reviewer 1 | 5 | 7.5 | 8 | 8 |
| Reviewer 2 | 5 | 7 | 7.5 | 8 |
| Mean | 5 | 7.25 | 7.75 | 8 |

### Example 5

Patients filled a validated questionnaire at the end of fourth and eighth sessions comprising two sections. The first section had four questions related to the effectiveness of the treatment rated on a scale of 0-5, with 0 (strongly disagree) and 5 (strongly agree).

Results of patient self-assessment questionnaire related to the efficacy of the treatment on a scale of 1-5.

TABLE 5

| Category | After 4 Sessions | After 8 Sessions |
|---|---|---|
| Growth of hair | 3 | 4 |
| Amount of noticeable | 2.5 | 4 |
| new hair | | |
| Reduction in visibility | 3 | 5 |
| of the scalp | | |
| Rate of hair loss | 3 | 4.5 |

### Example 6: Hair Pull Test

To assess the improvement in hair loss was done with the hair pull test. A bundle of 50-60 hairs were grasped between thumb, index finger, and middle finger and pulled from the base near to the scalp. Pulled out hair was count. Results were evaluated at baseline, after fourth session and eighth session.

### Example 7: Effect on Melanin Synthesis

The aim of this study was to evaluate the effect of the present composition on melanin synthesis and the expression of melanin production factors in B16F10 murine melanoma cells. Specifically, the study investigated whether the present composition enhanced melanin biosynthesis without causing cytotoxicity and increased mRNA levels of key melanin synthesis factors, thereby assessing its potential as a pigmenting agent.

### Preparation:

Murine melanoma cell line, B16F10 cells (ATCC, no. CRL-6475) maintained in DMEM medium (Dulbecco's Modified Ealge's Medium) containing 10% FBS (fetal bovine serum), penicillin (100 IU/ml), and streptomycin (100 μg/ml) at 37° C. in a humidified, 5% CO₂ atmosphere. The present composition was prepared in DMEM medium immediately before use.

### Protocol:

### 1. Evaluation of Melanin Contents

B16F10 cells were washed twice using DPBS (Dulbecco's Phosphate-Buffered Saline) and collected by centrifugation. Cell number was determined by trypan blue and resuspended the cells in DMEM medium. The cell suspension was dispensed into the wells of the 6-well plate to a final concentration of 2×10⁴ cells/well. The present composition was added and a cultured medium was used as an untreated control. The plate was incubated at 37° C. for 72 hours in a humidified, 5% CO₂ atmosphere. To measure the amount of intracellular melanin, the cultured cells were dissolved in 1 N NaOH containing 10% DMSO (dimethyl sulfoxide) at 60° C. for 1 hour. Record the absorbance at 490 nm using a 96-well plate reader.

The percentage of relative melanin contents was calculated as follows:

$$Relative melanin contents ( \% ) = ( absorbance of treatment / absorbance of non - treatment ) \times 100$$

2. Evaluation of mRNA Levels for Melanin Production Factors

B16F10 cells were washed twice using DPBS (Dulbecco's Phosphate-Buffered Saline) and collected by centrifugation. Cell number was determined by trypan blue and resuspended the cells in DMEM medium. The cell suspension was dispensed into the wells of the 6-well plate to a final concentration of 2×10⁴ cells/well. The present composition was added and a cultured medium was used as an untreated control. The plate was incubated at 37° C. for 24 hours in a humidified, 5% CO₂ atmosphere. The total RNA was extracted using TRI-solution (BSK, Korea) according to the manufacturer's instructions, and quantification by Take 3 microvolume plate (Agilent, USA). The complementary DNA was synthesized using PrimeScript Reverse Transcriptase (Takara, Japan). RT-PCR (reverse transcription polymerase chain reaction) was performed using a PCR thermocycler (Thermo Fisher Scientific, USA). The PCR products were examined after gel electrophoresis. Band images were quantified using Image J software.

The percentage of relative mRNA level was calculated as follows:

$$Relative mRNA level ( \% ) = ( Band intensity of treatment group / Band intensity of non - treatment group ) \times 100$$

### Results

### Effect of Present Composition on Melanin Synthesis:

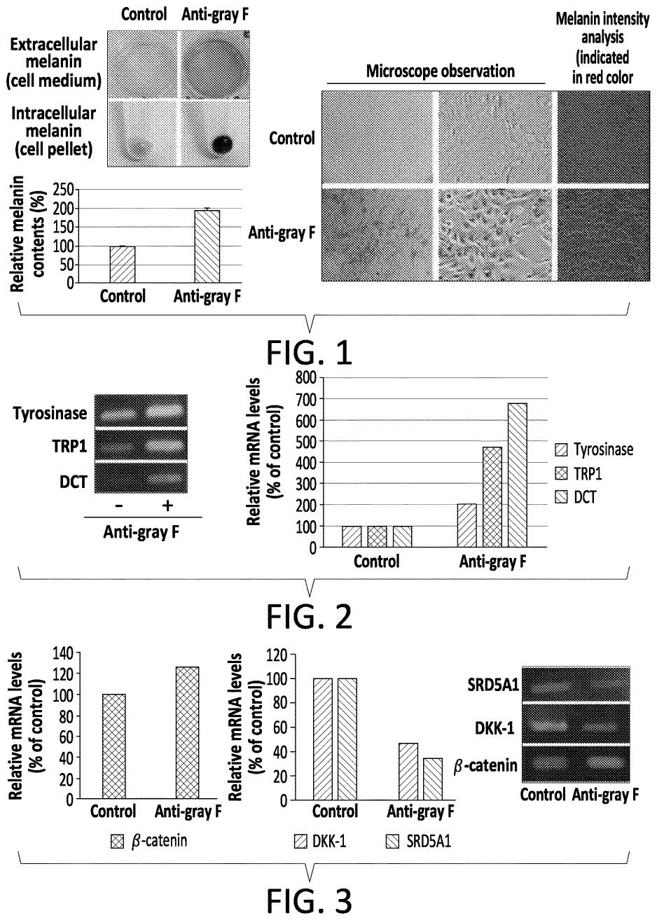

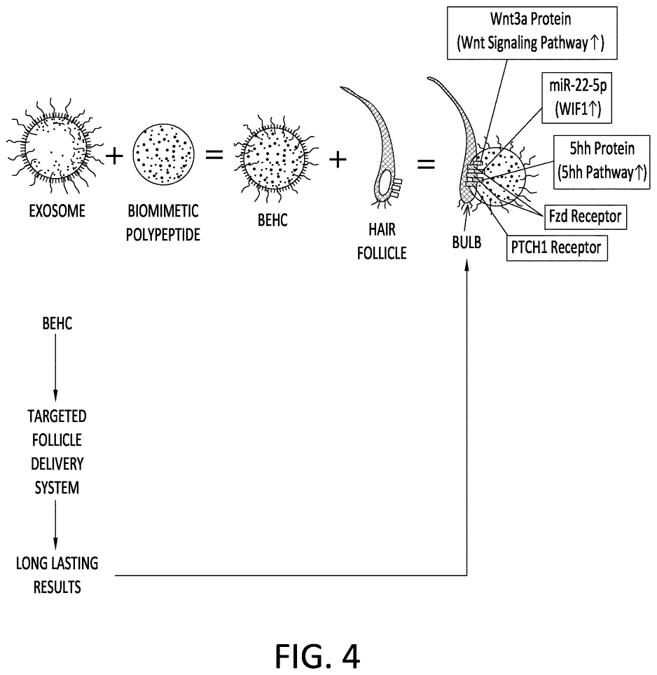

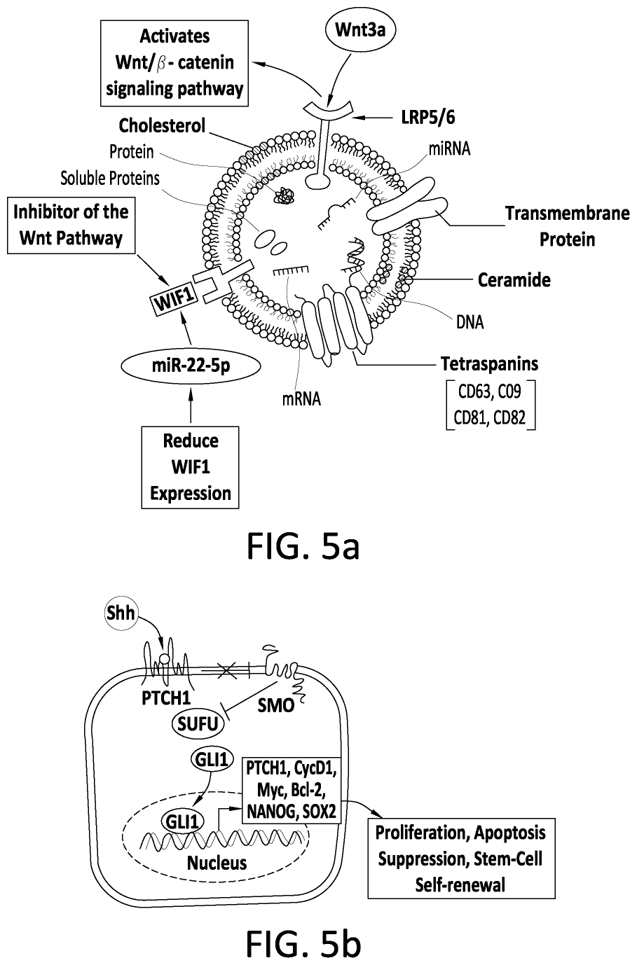

The results showed that the present composition significantly increases melanin biosynthesis in B16F10 cells compared to the control by over 200% relative melanin content without causing cytotoxicity (as depicted in FIG. 1). Therefore, present composition has an effect as a pigmenting agent.

Effect of Present Composition on the mRNA Levels of Melanin Synthesis Factors

The result indicated a notable elevation in the mRNA expression levels of melanin synthesis factors by 200% in tyrosinase, 475% in TRP1 and 675% in DCT expression following treatment with the present composition (as depicted in FIG. 2). Therefore, present composition has an effect as a pigmenting agent.

### Example 8: Effect of the Present Composition on the Regulation of Hair Follicle Cycle Factors

The aim of this study was to evaluate the effect of the present composition on the regulation of hair follicle cycle factors in human hair follicle dermal papilla cells (HFDPC). Specifically, the study aimed to assess whether the present composition could inhibit hair loss and stimulate hair growth by analyzing changes in the mRNA levels of key regulatory factors, including DKK-1, SRD5A1, and β-catenin.

Preparation: Hair follicle dermal papilla cells (HFDPC) (Promo Cell, lot. 485Z014.1) maintained in DMEM medium (Dulbecco's Modified Ealge's Medium) containing 10% FBS (fetal bovine serum), penicillin (100 IU/ml), and streptomycin (100 μg/ml) at 37° C. in a humidified, 5% CO2 atmosphere. The present composition was prepared in DMEM medium immediately before use.

### Protocol:

HFDPC were washed twice using DPBS (Dulbecco's Phosphate-Buffered Saline) and collected by centrifugation. Cell number was determined by trypan blue and resuspended the cells in DMEM medium. The cell suspension was dispensed into the wells of the 6-well plate to a final concentration of 2×10⁴ cells/well. The present composition was added and a cultured medium was used as an untreated control. The plate was incubated at 37° C. for 24 hours in a humidified, 5% CO2 atmosphere. The total RNA was extracted using TRI-solution (BSK, Korea) according to the manufacturer's instructions, and quantification by Take 3 microvolume plate (Agilent, USA). The complementary DNA was synthesized using PrimeScript Reverse Transcriptase (Takara, Japan). RT-PCR (reverse transcription polymerase chain reaction) was performed using a PCR thermocycler (Thermo Fisher Scientific, USA). The PCR products were examined after gel electrophoresis.

### Quantification:

Band images were quantified using Image J software, and the percentage of relative mRNA level was calculated as follows:

$$Relative mRNA level ( \% ) = ( Band intensity of treatment group / Band intensity of non - treatment group ) \times 100$$

### Result:

### Effect of the Present Composition on Inhibiting Hair Loss and Stimulating Hair Growth:

The effects of the present composition on the mRNA levels of factors involved in regulating the hair follicle cycle were assessed. The treatment group showed a reduction in mRNA levels of DKK-1 by 55% and SRD5A1 by 65% compared to the control group, while an 125% increase in expression level of β-catenin was observed (as depicted in FIG. 3). These findings indicate that the use of present composition may have the potential to inhibit hair loss and promote hair growth.

### Example 9: Composition of Bone Marrow Conditioned Media

The composition of the Bone Marrow Conditioned Media (BMCM) is shown in Table 6. The Bone Marrow Conditioned Media (BMCM) composition was prepared by adding the Sh-oligopeptides, Sh-polypeptides, and Oligopeptide-1 conjugates under continuous stirring at 750-1000 rpm at ambient temperature. The pH was adjusted to 6.8-7.2, followed by sterile filtration (0.22 μm) and aseptic filling into 1 ml vials.

The BMCM functioned as the primary bioactive driver, delivering naturally secreted cytokines, extracellular vesicles, and growth factors. These elements collectively:
Stimulated dermal papilla cell proliferation and metabolic activity,
Reinforced perifollicular extracellular matrix (ECM) integrity and signaling,
Promoted angiogenic support through VEGF-like pathways, and
Conferred potent antioxidant and cytoprotective effects within the follicular microenvironment.

TABLE 6

| Sr No. | Ingredient | Actual Weight | | Properties |
|---|---|---|---|---|
| 1 | Sh-Oligopeptide-1 | 0.2497 | mg/L | Skin |
| | | | | Conditioning |
| 2 | Sh-Polypeptide-43 | 0.2497 | mg/L | Skin |
| | | | | Conditioning |
| 3 | Sh-Polypeptide-72 | 0.2497 | mg/L | Skin |
| | | | | Conditioning |
| 4 | Sh-Oligopeptide-10 | 0.2497 | mg/L | Skin |
| | | | | Conditioning |
| 5 | Oligopeptide-1 | 249.656 | mg/L | Skin |
| | | | | Conditioning |
| 6 | sr-(Methionyl | 0.2497 | mg/L | Skin |
| | shOligopeptide-75 | | | Conditioning |
| | shPolypeptide-60 | | | |
| | Hexapeptide-40) | | | |
| 8 | Sh-polypeptide-14 | 0.5 | mg/L | Skin |
| | | | | Conditioning |
| 9 | Oligopeptide-1- | 24.9656 | mg/L | Skin |
| | Sh-Polypeptide-11 | | | Conditioning |
| 10 | Oligopeptide-1- | 24.9656 | mg/L | Skin |
| | ShPolypeptide-8 | | | Conditioning |
| 11 | Oligopeptide-1- | 49.931 | mg/L | Skin |
| | shpolypeptide-62 | | | Conditioning |

### Example 10

A randomized trial was conducted in 50 patients (18-55 years) with early to moderate AGA. The test group received the Bone Marrow Conditioned Media (BMCM) composition comprising Sh-oligopeptides, Sh-polypeptides, and Oligopeptide-1 conjugates via microneedling every three weeks for 21 weeks (8 sessions). The hair density (primary), hair shaft thickness and patient satisfaction (secondary), and safety were evaluated through local tolerability and systemic assessments at baseline and Week 21.

When the BMCM composition was combined with the hair growth and anti-hair greying composition it resulted in regeneration, accelerated anagen induction, and increased hair shaft thickening.

### Results:

Hair Growth Acceleration: The BMCM composition reduced the telogen-to anagen transition time, with visible new hair shafts emerging within 4-6 weeks, compared to 8-10 weeks for the control formulation.
Increased Hair Density: A 28-35% increase in hair density after 12 weeks, significantly superior to the base composition was observed.
Follicular Protection: Through anti-apoptotic and anti-stress factors inherent in BMCM, dermal papilla cells were shielded from oxidative and inflammatory insults, thereby limiting follicular miniaturization in androgenetic alopecia models.
Synergy with Peptides: BMCM when combined with the present hair growth and anti-hair greying composition showed potential activity resulting in greater efficacy.

Bone Marrow Conditioned Media (BMCM) activated the EGFR-mediated MAPK/ERK, PI3K/Akt, and JAK/STAT signalling pathways, thereby promoting keratinocyte and fibroblast proliferation, enhancing follicular survival, stimulating angiogenesis and ECM remodelling, collectively facilitating anagen initiation and restoration of scalp homeostasis. Sh-Polypeptide-14 activated TGF-β/SMAD, MAPK, and PI3K/Akt pathways, promoting ECM synthesis, immune regulation, and follicular resilience to sustain dermal regeneration and extend the anagen phase.

### Conclusion:

Thus, the Bone Marrow Conditioned Media composition enhanced hair growth stimulation and follicular protection. The addition of biomimetic peptide conjugates further improved Bone Marrow Conditioned Media composition's regenerative effects, thereby providing a formulation with superior therapeutic outcomes compared to the base composition or individual components alone.

### Example 11

A total of 112 male participants, aged 25-50, with advanced androgenetic alopecia were randomly divided into:
Group A: Received the biomimetic polypeptide formulation of Table 1 herein intradermally before transplant, used the biomimetic polypeptide formulation as the graft storage solution, and continued post-transplant therapy with the biomimetic polypeptide formulation.
Group B: Received a conventional hair transplant without the present biomimetic polypeptide formulations. Saline was used as the graft storage medium.

Assessments included global photographic scoring and videomicroscopy. Group A showed significantly higher scores across all metrics:
Higher density and graft survival
Greater terminal hair counts
Thicker hair shafts

This comparative structure allowed investigators to directly attribute improvements to the biomimetic polypeptide formulations disclosed herein, validating its dual function as both treatment and preservation tool.

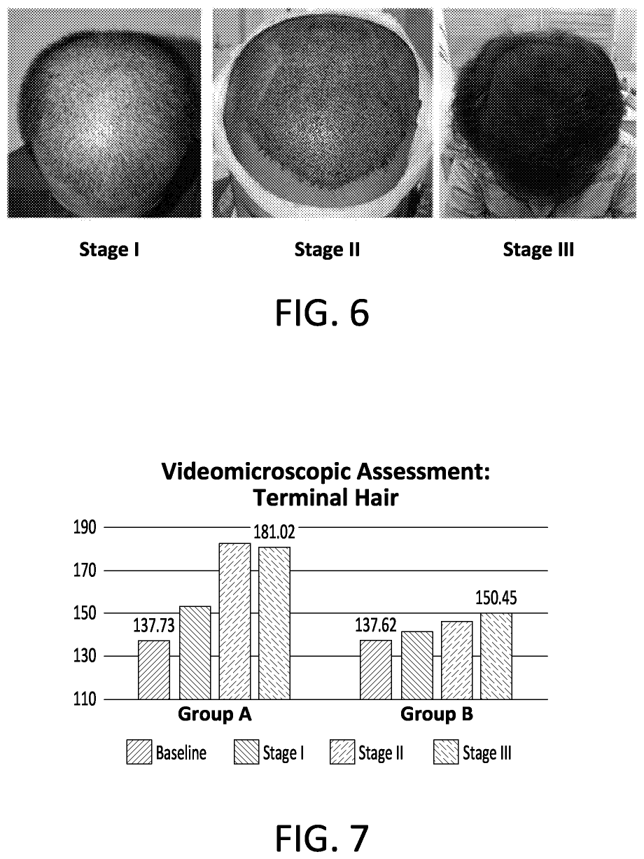

The study compared two groups: Group A (our biomimetic polypeptide formulations) and Group B (Conventional method). The results clearly showed superior outcomes in Group A across all key metrics. As seen in FIG. 6, patients in Group A showed increasing regrowth of hair. FIG. 6 shows hair regrowth progression at stages I, II and III during and after treatment with the biomimetic polypeptide formulations disclosed herein, in conjunction with hair transplant surgery.

Videomicroscopy is a high-resolution imaging technique that magnifies the scalp and hair to assess hair thickness, density, and treatment response in hair loss. Videomicroscopic assessments further confirmed the superior efficacy of our biomimetic polypeptide formulations over the conventional method. Group A (our biomimetic polypeptide formulations) demonstrated significant improvements from baseline to Stage III.

As seen in Table 7 and FIG. 7, patients in Group B, receiving a conventional hair transplant without the biomimetic polypeptide formulations disclosed herein, showed an increase in terminal hairs of about 9.3%. Patients in Group A, receiving a hair transplant in conjunction with the biomimetic polypeptide formulations, showed an increase in terminal hairs of about 31.4%.

TABLE 7
Terminal Hair

| Parameters | Baseline | Stage I | Stage II | Stage III |
|---|---|---|---|---|
| Group A | 137.73 | 153.44 | 182.67 | 181.02 |
| Group B | 137.62 | 141.42 | 146.37 | 150.45 |

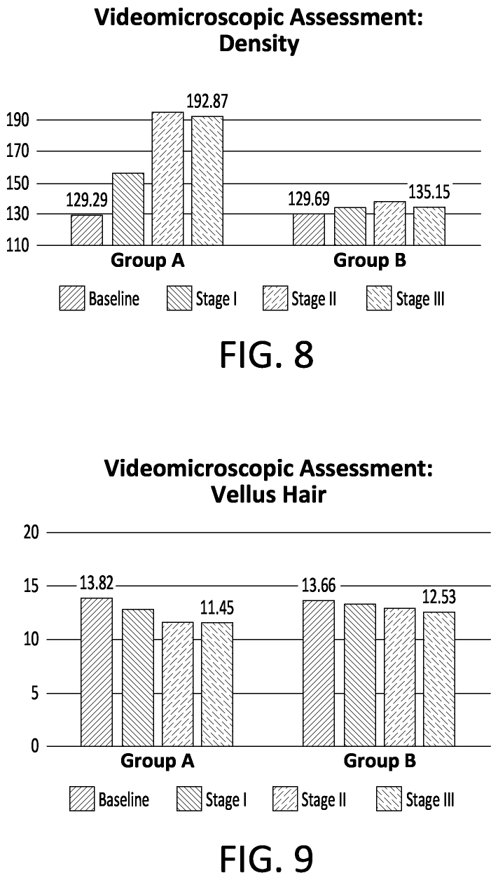

As seen in Table 8 and FIG. 8, patients in Group B showed little or no change in hair density, with hair density increasing by about 4.2%. Patients in Group A showed an increase in hair density of about 49.2%. Hair density evaluation revealed approximately 43% higher graft uptake in Group A compared to Group B, indicating superior graft viability with the disclosed biomimetic polypeptide formulations as the storage medium.

TABLE 8
Density

| Parameters | Baseline | Stage I | Stage II | Stage III |
|---|---|---|---|---|
| Group A | 129.29 | 156.38 | 195.78 | 192.87 |
| Group B | 129.69 | 134.36 | 138.2 | 135.15 |

As seen in Table 9 and FIG. 9, patients in Group A and Group B each showed a decrease in vellus hair hairs, with vellus hairs declining by 17% for the patients in Group A and 8.3% for the patients in Group B.

TABLE 9
Vellus Hairs

| Parameters | Baseline | Stage I | Stage II | Stage III |
|---|---|---|---|---|
| Group A | 13.82 | 12.81 | 11.63 | 11.45 |
| Group B | 13.66 | 13.27 | 12.79 | 12.53 |

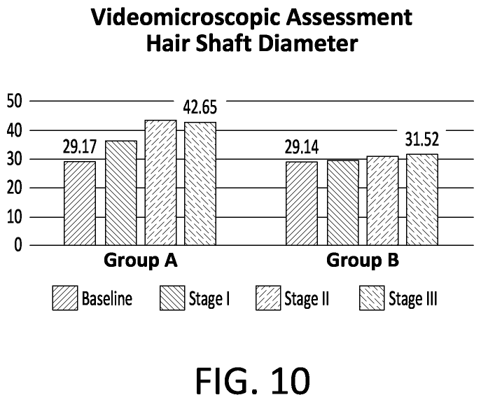

As seen in Table 10 and FIG. 10, patients in Group B, receiving a conventional hair transplant without the biomimetic polypeptide formulations disclosed herein, showed an increase in hair shaft diameter of about 8.2%. Patients in Group A, receiving a hair transplant in conjunction with the biomimetic polypeptide formulations, showed an increase in hair shaft diameter of about 46.2%.

TABLE 10
Hair Shaft Diameter

| Parameters | Baseline | Stage I | Stage II | Stage III |
|---|---|---|---|---|
| Group A | 29.17 | 36.15 | 43.22 | 42.65 |
| Group B | 29.14 | 29.67 | 30.9 | 31.52 |

Compared to Group B, Group A, the biomimetic polypeptide formulations group exhibited statistically significant gains in hair growth metrics at every stage, demonstrating not only the regenerative potential of the peptide formulation but also its viability in preserving graft quality and enhancing post-transplant outcomes. Changes in number of terminal hairs, hair density, and hair shaft diameter were statistically significant (p<0.001) but clinically transformative. our biomimetic polypeptide formulations showcased enhanced scalp conditioning, accelerated recovery, improved graft survival, and dramatically better aesthetic results compared to traditional methods. This comparative analysis confirms that the biomimetic polypeptide formulations outperform saline in preserving follicular viability and promoting post-transplant hair density. It provides a superior, clinically validated solution for androgenetic alopecia and hair restoration.

This comparative data shows that the biomimetic polypeptide formulations enhances the success rate of FUE transplants through biological preservation-providing clinicians and patients with a robust, clinically proven, and safe alternative to conventional storage methods like saline

We claim:

1. A hair growth and anti-hair greying composition comprising;
i. Peptides selected from Caffeoyl Tripeptide-1 in an amount of 0.05 mg/L to 10 mg/L; Stem Cell Factor (SCF) including its biomimetic variant sh-Polypeptide-4 in an amount of 0.05 mg/L to 15 mg/L; Caffeoyl sh-Decapeptide-9 in an amount of 0.05 mg/L to 10 mg/L; Tetrapeptide-32 in an amount of 0.05 mg/L to 15 mg/L; Superoxide Dismutase 1 (SOD-1) including its biomimetic variant sh-Polypeptide-60 in an amount of 0.05 mg/L to 15 mg/L; Sirtuin 1 (Sirt-1) including its biomimetic variant sh-Polypeptide-66 in an amount of 0.01 mg/L to 5 mg/L; Alanine/Histidine/Lysine Polypeptide Copper HCl [cu-AHK] in an amount of 0.5 mg/L to 50 mg/L; Biotinoyl sh-Oligopeptide-129 SP [BIO-TDP] in an amount of 10 mg/L to 75 mg/L; alone or mixtures thereof.
ii. Exosomes selected from Panax ginseng Adventitious Root Extracellular Vesicles in an amount of 0.5 mg/L to 50 mg/L; Human Umbilical Mesenchymal Stem Cell Conditioned Media in an amount of 50000 mg/L to 100000 mg/L alone or mixtures thereof.
iii. Hepatocyte Growth Factor (HGF) including its biomimetic variant Sh-Polypeptide 62 in an amount of 0.5 mg/L to 50 mg/L;
iv. Platelet-Derived Growth Factor A (PDGFA) including its biomimetic variant Sh-Polypeptide-8 and/or Sh-Polypeptide-59 in an amount of 0.5 mg/L to 500 mg/L;
along with
v. Vascular endothelial growth factor (Human oligopeptide-11) including its biomimetic analogue Sh-Polypeptide-9 in an amount of 0.01 mg/L-100 mg/L;
vi. Basic fibroblast growth factor (Human oligopeptide-3) including its biomimetic analogue Sh-Polypeptide-1 in an amount of 0.01 mg/L to100 mg/L;
vii. Insulin like growth factor (Human oligopeptide-2) including its biomimetic analogue Sh-Oligopeptide-2 in an amount of 0.01 mg/L to 100 mg/L;
viii. Copper tripeptide 1 in an amount of 0.1 mg/L to 500 mg/L;
ix. Keratinocyte growth factor including its biomimetic analogue Sh-Polypeptide-3 in an amount of 0.01 mg/L to 100 mg/L;
x. Thymosin β4 including its biomimetic analogue Sh-Oligopeptide-4 in an amount of 0.005 mg/L to 100 mg/L;
xi. Noggin including its biomimetic variant sh-Polypeptide-13 in an amount of 9 to 11 mg/L;
xii. Decapeptide-10 (CG-Keramin2) in an amount of 8.5 to 10.5 mg/L;
xiii. Octapeptide-2 (Prohairin-β4) in an amount of 0.0025 to 0.005 mg/L;
xiv. Vitamins, minerals, nucleic acids and amino acids in appropriate concentration;
and with pharmaceutical and dermatological acceptable ingredients and excipients.

2. The composition as claimed in claim 1, wherein the composition further comprises at least one peptide selected from the group consisting of Elastin scalp peptide (Tetrapeptide-32) in the concentration of about 20 ppm; Elastin Hair Peptide (Hexapeptide-12) in the concentration of about 20 ppm; Acetyl Tetrapetide-3 in an appropriate concentration; Octapeptide-11 in an appropriate concentration; Octapeptide-20 in an appropriate concentration; Palmitoyl pentapeptide-3 in an appropriate concentration; Oligopeptide-74 (CG-Grotene) in an appropriate concentration; Sh-Oligopeptide-72 in a concentration of 0.2497 mg/L; and Sh-Polypeptide-14 in a concentration of 0.5 mg/L.

3. The composition as claimed in claim 2, wherein the composition further comprises a bone marrow conditioned media formulation, said bone marrow conditioned media formulation comprising at least one compound selected from the group consisting of Sh-Oligopeptide-1 in a concentration of 0.2497 mg/L, Sh-Polypeptide-43 in a concentration of 0.2497 mg/L, Sh-Oligopeptide-10 in a concentration of 0.2497 mg/L, Oligopeptide-1 in a concentration of 249.656 mg/L, sr-(Methionyl ShOligopeptide-75 Sh-Polypeptide-60 Hexapeptide-40) in a 5 concentration of 24.96 mg/L, Oligopeptide-1-Sh-Polypepptide-11 in a concentration of 24.96 mg/L, Oligopeptide-1-Sh-Polypeptide-8 in a concentration of 24.96 mg/L, Oligopeptide-1-Sh-Polypeptide-62 in a concentration of 49.93 mg/L.

4. The composition as claimed in claim 1, wherein the vitamins are selected from the group consisting of Vitamin A in an amount of 0.01 to 0.50 mg/L, Vitamin B1 in an amount of 0.01 to 0.50 mg/L, Vitamin B2 in an amount of 0.01 to 0.50 mg/L, Vitamin B3 in an amount of 1.0 to 50 mg/L, Vitamin B5 in an amount of 0.001 to 0.050 mg/L, Vitamin B6 in an amount of 0.01 to 0.10 mg/L, Vitamin B7 in an amount of 0.001 to 0.050 mg/L, Vitamin B12 in an amount of 0.01 to 0.50 mg/L, Vitamin C in an amount of 10 to 80 mg/L, Vitamin E in an amount of 0.001 to 0.050 mg/L, and Vitamin K in an amount of 0.001 to 0.050 mg/L.

5. The composition as claimed in claim 1, wherein the minerals and mineral salts are selected from the group consisting of a calcium salt in an amount of 100 to 300 mg/l, a sodium salt in an amount of 5000 to 7000 mg/l, a potassium salt in an amount of 200 to 600 mg/l, and a magnesium salt in an amount of 10 to 200 mg/L.

6. The composition as claimed in claim 1, wherein:
the nucleic acids are selected from the group consisting of adenosine, cytosine, guanine and thymine in an amount of 1-50 mg/l; and
the essential and non-essential amino acids are selected from the group consisting of taurine, hydroxyproline, methionine, proline, ornithine, asparagine, glutamic acid, aspartic acid, cystine, serine, glycine, tyrosine, phenylalanine, isoleucine, tryptophan, threonine, leucine, histidine, valine, arginine, lysine, alanine and glutamine in an amount of 2-150 mg/L.

7. The composition as claimed in claim 1, wherein the pharmaceutically and dermatologically acceptable excipients are selected from the group consisting of diluents, carriers, buffering agents, humectant, and colorants in appropriate concentrations.

8. The composition as claimed in claim 1, wherein the composition further comprises a growth factor selected from the group consisting of follistatin, Wnt proteins, β catenin proteins, stem cell factor, and platelet derived growth factors in an appropriate concentration.

9. The composition as claimed in claim 1, wherein the composition further comprises a hair growth promoting compound selected from the group consisting of minoxidil, minoxidil analogues, minoxidil derivatives, anti-androgens and 5-alpha-reductase inhibitors.

10. The composition as claimed in claim 1, wherein said composition is formulated as an intradermal or topical composition.

11. The composition as claimed in claim 1, wherein said composition is formulated as ready-to-inject sterile glass vial, as powder-liquid composition in two separate glass vials, as prefilled syringes or combined with Hyaluronic Acid.

12. A hair growth and anti-hair greying composition prepared by the process comprising:
i. placing distilled water at ambient temperature in a disperser set at 750-1000 rpm;
ii. adding at least one peptide to the distilled water to form a mixture, wherein the peptide is selected from the group consisting of caffeoyl tripeptide-1 in an amount of 0.05 mg/L to 10 mg/L; stem cell factor (SCF) including its biomimetic variant sh-polypeptide-4 in an amount of 0.05 mg/L to 15 mg/L; caffeoyl sh-decapeptide-9 in an amount of 0.05 mg/L to 10 mg/L; tetrapeptide-32 in an amount of 0.05 mg/L to 15 mg/L; superoxide dismutase 1 (SOD-1) including its biomimetic variant sh-polypeptide-60 in an amount of 0.05 mg/L to 15 mg/L; sirtuin 1 (Sirt-1) including its biomimetic variant sh-polypeptide-66 in an amount of 0.01 mg/L to 5 mg/L; alanine/histidine/lysine polypeptide copper HCl [cu-AHK] in an amount of 0.5 mg/L to 50 mg/L; biotinoyl sh-oligopeptide-129 SP [BIO-TDP] in an amount of 10 mg/L to 75 mg/L;
iii. adding to the mixture at least one exosome selected from the group consisting of Panax ginseng Adventitious Root Extracellular Vesicles in an amount of 0.5 mg/L to 50 mg/L and Human Umbilical Mesenchymal Stem Cell Conditioned Media in an amount of 50,000 mg/L to 100000 mg/L;
iv. adding to the mixture at least one Growth factor selected from the group consisting of Hepatocyte Growth Factor (HGF) including its biomimetic variant Sh-Polypeptide 62 in an amount of 0.5 mg/L to 50 mg/L; Platelet-Derived Growth Factor A (PDGFA) including its biomimetic variant Sh-Polypeptide-8 and/or Sh-Polypeptide-59 in an amount of 0.5 mg/L to 500 mg/L; Vascular endothelial growth factor (Human oligopeptide-11) including its biomimetic analogue Sh-Polypeptide-9 in an amount of 0.01 mg/L-100 mg/L; Basic fibroblast growth factor (Human oligopeptide-3) including its biomimetic analogue Sh-Polypeptide-1 in an amount of 0.01 mg/L-100 mg/L; Insulin like growth factor (Human oligopeptide-2) including its biomimetic analogue Sh-Oligopeptide-2 in an amount of 0.01 mg/L-100 mg/L; Copper tripeptide 1 in an amount of 0.1 mg/L-500 mg/L; Keratinocyte growth factor including its biomimetic analogue Sh-Polypeptide-3 in an amount of 0.01 mg/L-100 mg/L; Thymosin β4 including its biomimetic analogue Sh-Oligopeptide-4 in an amount of 0.005 mg/L-100 mg/L; Noggin including its biomimetic variant sh-Polypeptide-13 in an amount of 9-11 mg/L, Decapeptide-10 (CG-Keramin2) in an amount of 8.5-10.5 mg/L and Octapeptide-2 (Prohairin-β4) in an amount of 0.0025 to 0.005 mg/L; and mixtures thereof;
v. adding to the mixture vitamins, minerals, nucleic acids and amino acids in appropriate concentration; along with pharmaceutically and dermatologically acceptable excipients;
vi. stirring the mixture in the disperser to obtain a product;
vii. Adjusting the pH of the product and filtering the product to obtain the hair growth and anti-hair greying composition; and
viii. optionally filling vials with the hair growth and anti-hair greying composition.

13. The composition as claimed in claim 12, wherein the composition comprises at least one of:
a vitamin selected from the group consisting of Vitamin A in an amount of 0.01 to 0.50 mg/L, Vitamin B1 in an amount of 0.01 to 0.50 mg/L, Vitamin B2 in an amount of 0.01 to 0.50 mg/L, Vitamin B3 in an amount of 1.0 to 50 mg/L, Vitamin B5 in an amount of 0.001 to 0.050 mg/L, Vitamin B6 in an amount of 0.01 to 0.10 mg/L, Vitamin B7 in an amount of 0.001 to 0.050 mg/L, Vitamin B12 in an amount of 0.01 to 0.50 mg/L, Vitamin C in an amount of 10 to 80 mg/L, Vitamin E in an amount of 0.001 to 0.050 mg/L, Vitamin K in an amount of 0.001 to 0.050 mg/L;
a mineral or mineral salt selected from the group consisting of a calcium salt in an amount of 100 to 300 mg/l, a sodium salt in an amount of 5000 to 7000 mg/l, a potassium salt in an amount of 200 to 600 mg/l, and a magnesium salt in an amount of 10 to 200 mg/L;
a nucleic acid selected from the group consisting of adenosine, cytosine, guanine and thymine in an amount of 1-50 mg/l; and
an amino acid selected from the group consisting of taurine, hydroxyproline, methionine, proline, ornithine, asparagine, glutamic acid, aspartic acid, cystine, serine, glycine, tyrosine, phenylalanine, isoleucine, tryptophan, threonine, leucine, histidine, valine, arginine, lysine, alanine and glutamine in an amount of 2-150 mg/L.

14. The composition as claimed in claim 12, wherein the pharmaceutically and dermatologically acceptable excipients are selected from the group consisting of diluents, carriers, buffering agents, humectant, colorants in an appropriate concentration.

15. The composition as claimed in claim 12, wherein the composition further comprises a bone marrow conditioned media formulation, said bone marrow conditioned media formulation comprising at least one peptide selected from the group consisting of Elastin scalp peptide (Tetrapetide 32) in the concentration of about 20 ppm; Elastin Hair Peptide (Hexapeptide-12) in the concentration of about 20 ppm; Acetyl Tetrapetide-3 in appropriate concentration; Octapeptide-11 in appropriate concentration; Octapeptide-20 in appropriate concentration; Palmitoyl pentapeptide-3 in appropriate concentration; Oligopeptide-74 (CG-Grotene) in appropriate concentration; Sh-Oligopeptide-72 in a concentration of 0.2497 mg/L; and Sh-Polypeptide-14 in a concentration of 0.5 mg/L; Sh-Oligopeptide-1 in a concentration of 0.2497 mg/L, Sh-Polypeptide-43 in a concentration of 0.2497 mg/L, Sh-Oligopeptide-10 in a concentration of 0.2497 mg/L, Oligopeptide-1 in a concentration of 249.656 mg/L, sr-(Methionyl ShOligopeptide-75 Sh-Polypeptide-60 Hexapeptide-40) in a 5 concentration of 24.96 mg/L, Oligopeptide-1-Sh-Polypepptide-11 in a concentration of 24.96 mg/L, Oligopeptide-1-Sh-Polypeptide-8 in a concentration of 24.96 mg/L, and Oligopeptide-1-Sh-Polypeptide-62 in a concentration of 49.93 mg/L.

16. A method of treating hair loss by stimulating hair follicles and promoting hair growth and improving the bodily appearance of a person, comprising administering the hair growth and anti-hair greying composition as claimed in claim 1 in an effective amount to a person in need thereof.

17. A method for treating or reversing greying of hair and improving the bodily appearance of a person, comprising administering the pharmaceutical and dermatological hair growth and anti-hair greying composition as claimed in claim 1 in an effective amount to a person in need thereof.

18. The method as claimed in claim 16, wherein:
said composition is administered intradermally or applied topically; or
said composition is co-administered with a hair growth promoting compound selected from the group consisting of minoxidil, minoxidil analogs, minoxidil derivatives, anti-androgens, and 5-alpha-reductase inhibitors.

19. A kit comprising;
a) prefilled syringes or vials or a dermaroller comprising the pharmaceutical or dermatological composition as claimed in claim 1 or hair filler insert embedded in hyaluronic acid enriched with composition of claim 1; and
b) instruction manual for application.

20. A bioengineered exosomal hair growth factor complex (BEHCTM) for promoting hair follicle growth and regeneration by activating Wnt3a protein, suppressing WIF1 and activating the Sonic Hedgehog (Shh) signaling pathway, comprising:
i. Exosomes in a size range of 50-150 nm; and
ii. Biomimetic polypeptides selected from the group consisting of Sh-Polypeptide-9 (VEGF); Sh-Polypeptide-1 (bFGF); Sh-Oligopeptide-2 (IGF-1); Sh-Polypeptide-3 (KGF); Sh-Oligopeptide-4 (Thymosin β4); Copper Tripeptide-1; Hepatocyte Growth Factor (HGF); Stem Cell Factor (SCF); and Noggin.

21. A method for promoting hair follicle growth and regeneration in a patient in need thereof by activating Wnt3a protein, suppressing WIF1 and activating the Sonic Hedgehog (Shh) signaling pathway, comprising:
i. administering the bioengineered exosomal hair growth factor complex system to the patient via injection, or through microneedling; and
ii. enabling targeted delivery to the hair bulb and activation of regenerative pathways.
